(19) United States
(12) Patent Application Publication
(10) Pub. No.: US 2023/0114400 A1
(43) Pub. Date: Apr. 13, 2023

(54) POROUS SUBSTRATE-BASED DIAGNOSTIC DEVICES

(71) Applicant: New York University in Abu Dhabi Corporation, Abu Dhabi (AE)

(72) Inventors: Mohammad A. Qasaimeh, Abu Dhabi (AE); Pavithra Sukumar, Abu Dhabi (AE); Muhammedin Deliorman, Abu Dhabi (AE)

(21) Appl. No.: 18/045,208

(22) Filed: Oct. 10, 2022

Related U.S. Application Data
(60) Provisional application No. 63/253,997, filed on Oct. 8, 2021.

(51) Int. Cl. G01N 33/543; G01N 33/569

(52) U.S. Cl. CPC G01N 33/54391; G01N 33/56983

(57) ABSTRACT

The present invention provides microfluidic porous substrate-based devices for multiplexed biosensing. The devices are suitable for detecting viruses and bacteria, such as by way of detecting pathogenic genes and antibodies. The devices support reverse transcriptase loop-mediated isothermal amplification for rapid results within minutes. The devices also support vertical-lateral-vertical direction flow assays, such as in the form of a multi-layered adhesive bandage.

### CROSS-REFERENCE TO RELATED APPLICATIONS

The present application claims priority to U.S. Provisional Application No. 63/253,997, filed Oct. 8, 2021, which is incorporated by reference herein in its entirety.

### BACKGROUND OF THE INVENTION

The current COVID-19 pandemic, caused by the viral pathogen SARS-CoV-2, urged the need for development of simple, rapid, and low-cost point of care (POC) diagnostic devices. Currently, the gold standard for the detection of viral gene targets is reverse transcription quantitative PCR (RT-qPCR), which requires long test-to-result turnaround time, high-skilled professionals, and well-equipped facilities (Thi V et al., Sci Trans Med, 12, ebac7075, 2020). To overcome these drawbacks, adopting isothermal nucleic acid amplification (LAMP) emerges as a promising alternative for SARS-CoV-2 testing, as well as other pathogens. For example, recently a portable 3D-printed microfluidic POC device was reported to detect SARS-CoV-2 genes in 40-60 minutes using RT-LAMP assay (Anurup G et al., PNAS, 117, 37,22727, 2020). However, it is still imperative to seek approaches that will overcome the paper-based RT-LAMP assay limitations.

At the time of infection, and during its different stages, the first line of defense against SARS-CoV-2 is the immune response, which includes the production of immunoglobulin M (IgM) and immunoglobulin G (IgG) antibodies in the blood (Long, Q. X., et al., 2020, Nature Medicine, 26:845-848; Dispinseri, S., et al., 2021, Nature Communications, 12:2670). In the process, IgM and IgG antibodies inhibit the viral load by binding to spike (S) and nucleocapsid (N) proteins of SARS-CoV-2 (Barnes, C. O., et al., 2020, Cell, 182(4):828-842; Ye, Q., et al., 2021, Frontiers in Immunology, 12:719037). In symptomatic individuals, IgM antibodies appear in the early stages of viral infection and are therefore an important indicator of the peak infection period. IgG antibodies, on the other hand, replace IgM antibodies after onset of symptoms, and are therefore essential for long-term immunity and immunological memory (16, 17 Li, P., et al., 2021, Journal of Clinical Laboratory Analysis, 26:e24080; Guo, L., et al., 2020, Clinical Infectious Diseases, 71(15):778-785). Therefore, it becomes important to dynamically monitor serum IgM and IgG antibodies for efficient diagnosis and screening of SARS-CoV-2 infections, both in symptomatic and asymptomatic individuals (Long, Q. X., et al., 2020, Nature Medicine, 26:845-848; Lei, Q., et al., 2021, Allergy, 76(2):551-561; Jiang, C., et al., 2020, Clinical & Translational Immunology, 9(9):e1182).

Thus, there is a need in the art for cost-effective, user-friendly, and self-powered testing POC devices. The present invention meets this need.

### SUMMARY OF THE INVENTION

In one aspect, the present invention relates to a porous substrate-based diagnostic device, comprising: a sheet of porous substrate having a thickness; and a hydrophobic material patterned on the sheet of porous substrate; wherein the hydrophobic material pattern permeates through the thickness of the sheet of porous substrate such that the sheet of porous substrate comprises a plurality of hydrophilic regions divided by the hydrophobic material; and wherein the hydrophobic material pattern defines a central hydrophilic sample region fluidly connected to at least one hydrophilic test region.

In one embodiment, the porous substrate is paper, cloth, woven fabrics, non-woven fabrics, threads, yarns, or any other porous materials or perforated films including acrylamide polymers or polyvinyl alcohol.

In one embodiment, the hydrophobic material is wax, a silane, a siloxane, a silanized glass, a fluororesin, or a silicone resin. In one embodiment, at least one hydrophilic test region comprises a capture molecule or probe. In one embodiment, the capture molecules or probes are antibodies, antibody fragments, antigens, aptamers, bacteriophage, proteins, nucleic acids, oligonucleotides, DNA molecules, RNA molecules, peptides, lipids, lectins, inhibitors, activators, ligands, hormones, cytokines, sugars, amino acids, fatty acids, phenols, or alkaloids.

In one embodiment, the at least one hydrophilic test region is arranged around the central hydrophilic sample region in a radial pattern. In one embodiment, the at least one hydrophilic test region is arranged laterally with the sample region.

In one embodiment, the central hydrophilic sample region is configured to receive a liquid sample. In one embodiment, the device is configured to detect the presence of one or more nucleic acid molecule associated with a disease or disorder in the liquid sample. In one embodiment, the device is configured to support one or more loop mediated isothermal amplification (LAMP) assays.

In one aspect, the present invention relates to methods of detecting one or more target molecule in a liquid sample. In one embodiment, the one or more target molecule are nucleic acid molecules. In one embodiment, the target molecules are DNA or RNA molecules. In one embodiment, the one or more target molecule is a diagnostic biomarker. In one embodiment, the one or more target molecule is a DNA or RNA molecule associated with a disease or disorder. In one embodiment, the liquid sample is a biological sample from a subject.

In one embodiment, the method comprises the steps of collecting a sample from a subject and/or patient, introducing the sample to the receiving or sample area of a device of the present disclosure, and interpreting the results displayed in the one or more test chambers and/or detection zones.

In one embodiment, the method comprises the steps of collecting a sample from a subject and/or patient, introducing the sample to the receiving or sample area of a device of the present disclosure, performing loop mediated isothermal amplification (LAMP) on the sample using at least one set of primers specific for detecting a target nucleic acid molecule, and interpreting the results displayed in the one or more test chambers and/or detection zones.

In one aspect, the present invention relates to methods of detecting one or more target nucleic acid molecule in a liquid sample. In one embodiment, the target nucleic acid molecule is associated with a disease or disorder.

In one aspect, the present invention relates to methods of diagnosing a disease or disorder in a subject, the method comprising the steps of collecting a sample from a subject and/or patient, introducing the sample to the receiving or sample area of a device of the present disclosure, performing loop mediated isothermal amplification (LAMP) on the sample using at least one set of primers specific for detecting a disease associated nucleic acid molecule, and interpreting the results displayed in the one or more test chambers and/or detection zones, and diagnosing the subject with a disease or disorder based on detection of the disease associated nucleic acid molecule. In one embodiment, the method further comprises administering a therapeutic agent for the treatment of the diagnosed disease or disorder.

In one aspect, the present invention relates to a vertical flow porous substrate-based diagnostic device, comprising: a plurality of sheets of porous substrate stacked on top of each other, each sheet of porous substrate having a thickness; wherein one or more porous layers comprises a hydrophobic material attached thereto to support a vertical flow assay, wherein the hydrophobic material permeates through the thickness of an individual porous layer, thereby dividing the layer into hydrophilic regions fluidly separated by hydrophobic barriers.

In one embodiment, the porous substrate is paper, cloth, woven fabrics, non-woven fabrics, threads, yarns, or any other porous materials or perforated films including acrylamide polymers or polyvinyl alcohol.

In one embodiment, the hydrophobic material is wax, a silane, a siloxane, a silanized glass, a fluororesin, or a silicone resin.

In one embodiment, a first sheet of porous substrate is configured to receive a liquid sample. In one embodiment, the first sheet of porous substrate is adjacent to one or more second porous layers, which comprise one or more hydrophilic test regions. In one embodiment, the liquid sample is configured to flow laterally between hydrophobic regions of adjacent sheets of porous substrates. In one embodiment, the liquid sample is configured to flow vertically through hydrophilic test regions across a sheet of porous substrate. In one embodiment, the one or more hydrophilic tests region comprise one or more capture molecules or probes. In one embodiment, the one or more capture molecules or probes are nanoparticles, antibodies, antibody fragments, antigens, aptamers, bacteriophages, proteins, nucleic acids, oligonucleotides, peptides, lipids, lectins, inhibitors, activators, ligands, hormones, cytokines, sugars, amino acids, fatty acids, phenols, and alkaloids. In one embodiment, the one or more capture molecules or probes are nanoparticles conjugated to one or more antigens targeted by one or more antibodies. In one embodiment, the one or more second porous layers are adjacent to one or more third porous layers on the face opposing the first porous layer, wherein the one or more third porous layer comprises one or more hydrophilic spots surrounded by hydrophobic barriers. In one embodiment, the one or more hydrophilic spots of the one or more third porous layers are aligned with the one or more hydrophilic test regions of the one or more second layers such that the liquid sample flows vertically from the test regions of the one or more second layers to the one or more hydrophilic spots of the one or more third layers.

In one embodiment, the device is configured to detect the presence of one or more target molecule in the liquid sample.

In one embodiment, the first sheet of porous substrate comprises a filter membrane. In one embodiment the thickness of the filter membrane is 0.1 mm to 1 mm.

In one embodiment, the one or more second layers comprise glass fibers, polyester fibers, nitrocellulose fibers, polyvinylidene difluoride or any perforated films. In one embodiment, the one or more second layers comprise one or more silanized glass fiber layers with hydrophilic regions of non-silanized glass. In one embodiment, the one or more third layers comprise one or more layers of nitrocellulose with hydrophilic regions surrounded by hydrophobic barriers.

In one embodiment, the device is in the form of an adhesive bandage such that the first sheet is configured to contact a wound. In some embodiments, the first sheet comprises at least one biodegradable porous microneedle for finger pricking via in-situ puncturing.

In one aspect, the present invention relates to methods of detecting one or more target molecule in a liquid sample. In one embodiment, the one or more target molecule are nucleic acid molecules including DNA and RNA molecules, antibodies, antigens, metabolites, or small molecules. In one embodiment, the one or more target molecule is a diagnostic biomarker. In one embodiment, the one or more target molecule is an antigen or antibody associated with a disease or disorder. In one embodiment, the liquid sample is a biological sample from a subject.

In one embodiment, the method comprises the steps of collecting a sample from a subject and/or patient, introducing the sample to the receiving or sample area of a device of the present disclosure, and interpreting the results displayed in the one or more test chambers and/or detection zones.

In one embodiment, the device is a packaged point-of-care diagnostic device for at-home use.

In one embodiment, the method comprises the steps of pricking the skin of a subject and/or patient such that a drop of blood is exuded from the pinprick, applying a device of the present disclosure to the skin of the subject and/or patient such that the drop of blood enters the receiving or sample area of the device, and interpreting the results displayed in the one or more test chambers and/or detecting zones.

In one embodiment, the method comprises the steps of applying a device of the present disclosure to the skin of the subject and/or patient, wherein the device comprises at least one biodegradable microneedle, such that the at least one biodegradable microneedle punctures the skin of the subject drop allowing at least a drop of blood to enter the receiving or sample area of the device, and interpreting the results displayed in the one or more test chambers and/or detecting zones.

In one aspect, the present invention relates to methods of diagnosing a disease or disorder in a subject, the method comprising the steps of collecting a sample from a subject and/or patient, introducing the sample to the receiving or sample area of a device of the present disclosure, wherein the device comprises at least one capture antigen or probe specific for detection of a disease associated biomarker, and interpreting the results displayed in the one or more test chambers and/or detection zones, and diagnosing the subject with a disease or disorder based on detection of the disease associated biomarker. In one embodiment, the method further comprises administering a therapeutic agent for the treatment of the diagnosed disease or disorder.

In one embodiment, the invention relates to a system comprising:

a) a first device comprising a porous substrate-based diagnostic device, comprising:

a sheet of porous substrate having a thickness; and

a hydrophobic material patterned on the sheet of porous substrate;

wherein the hydrophobic material pattern extends through the thickness of the sheet of porous substrate such that the sheet of porous substrate comprises a plurality of hydrophilic regions divided by the hydrophobic material; and

wherein the hydrophobic material pattern defines a central hydrophilic sample region fluidly connected to at least one hydrophilic test region; and

b) a second device comprising a vertical flow porous substrate-based diagnostic device, comprising:

a plurality of sheets of porous substrate stacked on top of each other, each sheet of porous substrate having a thickness and a hydrophobic material patterned on the sheet of porous substrate;

wherein the hydrophobic material pattern extends through the thickness of each sheet of porous substrate such that each sheet of porous substrate comprises a plurality of hydrophilic test regions divided by the hydrophobic material.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A through FIG. 1D, depicts representative images and data of the RT-LAMP assay. FIG. 1A depicts images of representative results of a conventional in-solution RT-LAMP assay for Gene N detection with a positive reaction containing 10⁴ copies/μL SARS-CoV-2 synthetic RNA and a No Template Control (NTC). The reactions were incubated for 40 minutes at 65° C. on a heating block at 300 rpm shaking. FIG. 1B depicts representative images of a paper-based RT-LAMP assay containing 10⁴ copies/μL SARS-CoV-2 synthetic RNA and an NTC. FIG. 1C depicts a representative short-range concentration gradient graph using of diffusion within the porous substrate. The inset depicts the COMSOL image of the gradient. FIG. 1D depicts representative printed wax hydrophobic barriers of confined microspots showing representative results of NPC, NTC, and Sample (RNA) of a pH-based assay and enhanced detection using in-situ gold nanoparticle aggregation, multiplex detection of NPC, actin primers (control), N gene, and E gene.

FIG. 2, comprising FIG. 2A through FIG. 2C, depicts representative imagine of conventional in-solution RT-LAMP detection of synthetic SARS-CoV-2 RNA and optimization parameters. FIG. 2A depicts representative imaging of in-solution RT-LAMP assays for determining the Limit of Detection (LoD) of Gene E with SARS-CoV-2 RNA titrated with serial log-10 dilutions and two negative controls, NTC and NPC. Reactions were incubated for 60 minutes at 65° C. FIG. 2B depicts representative imaging of in-solution RT-LAMP assays for determining the optimal temperature for Gene E primers from a range of 56-67° C. in a thermocycler. A camera photograph was taken every 10 minutes. The reaction volume was 25 μL for all experiments. Data are representative of at least three independent experiments. FIG. 2C depicts representative images of premixed RT-LAMP reactions in tubes (25 μL reaction volume) loaded on 5 mm×5 mm membranes of various materials. Gene E detection was performed with a positive reaction and NPC, each containing 10⁶ copies/4, SARS-CoV-2 synthetic RNA and an NTC for each membrane type. Samples were incubated at 65° C. in an oven in sterile sealed Petri dishes. Glass Fiber: Whatman™ Standard 14 sample pads; Grade 4: Whatman™ Grade No. 4 filter paper; NC-90: Vivid™ 90 Nitrocellulose; NC-120: Vivid™ 120 Nitrocellulose; PSM-GF, -GR, and -GX: Vivid™ Plasma Separation Membrane (PES)-GF, -GR, and -GX.

FIG. 3 depicts representative images of temperature optimization studies for in-solution RT-LAMP detection of Gene N using synthetic SARS-CoV-2 RNA. The optimal RT-LAMP assay temperature for Gene N primers was evaluated over a range of 56-67° C. in a thermocycler. Camera photographs were taken at 10-minute intervals. The reaction volume was 25 μL.

FIG. 4, comprising FIG. 4A through 4D, depicts representative images of paper-based (cellulose grade 4) RT-LAMP detection assays of synthetic SARS-CoV-2 RNA and optimization of parameters. FIG. 4A depicts representative images of LoD studies for the detection of gene E on 5 mm×5 mm grade 4 pads, loaded with 25 μL RT-LAMP reaction solution premixed in microcentrifuge tubes. Experiments were performed in an over at 65° C. for 60 minutes. FIG. 4B depicts representative images of optimal reaction volume studies for 5 mm diameter spots, determined by loading different reaction volumes and observing leakage from the spot. Positive samples for gene N detection were 10⁴ copies/μL SARS-CoV-2 synthetic RNA, heated at 65° C. FIG. 4C depicts representative schematics of laminating the bottom of chips with hydrophobic barriers (wax printed) and performing the assay on a hotplate. FIG. 4D depicts representative images of Time to Result (TtR) studies of the reactions on singleplex chips at a range of temperatures for gene N. Synthetic RNA was used at a concentration of 10⁴ copies/μL SARS-CoV-2 synthetic RNA for positive RNA and NPC samples, with a reaction volume of 8 Data are representative of at least three replicates.

FIG. 5, comprising FIG. 5A through 5D, depicts representative images of studies for the design and optimization of hydrophobic barriers by wax printing of AutoCAD designs. FIG. 5A depicts schematic representations of cellulose paper with hydrophobic patterning. Wax diffuses vertically upon heating for three minutes on a hotplate. FIG. 5B depicts representative images of spots with different barrier line thicknesses. FIG. 5C depicts representative front-side spot diffusion diameter of spots with different line thicknesses prepared at different temperatures. FIG. 5D depicts representative back-side spot diffusion diameter of spots with different line thicknesses prepared at different temperatures. Wax was not fully transferred vertically for spots heated at 30° C., 60° C., and line thicknesses of 0.05 mm, 0.15 mm, and 0.25 mm when heated at 90° C. Data represents mean±S.D.; n=3.

FIG. 6, comprising FIG. 6A through 6D, depicts representative images and data from temperature optimization studies for paper-based RT-LAMP detection of synthetic SARS-CoV-2 RNA. FIG. 6A depicts representative images of paper-based RT-LAMP detection of Gene E when reactions were performed at a range of 56-68° C. for 4 minutes with a concentration of 10⁴ copies/μL RNA template. FIG. 6B depicts representative images of paper-based RT-LAMP detection of Gene N when reactions were performed at a range of 56-68° C. for 4 minutes with a concentration of 10⁴ copies/μL RNA template. FIG. 6C depicts quantification of the mean intensity of the yellow color on paper from FIG. 6A. FIG. 6D depicts quantification of the mean intensity of the yellow color on paper from FIG. 6B. Data are presented as mean±D. D.; n=3.

FIG. 7, comprising FIG. 7A through FIG. 7D, depicts representative imaging of the enhanced detection of synthetic SARS-CoV-2 RNA with in-situ gold nanoparticle synthesis for tube-based colorimetric RT-LAMP. FIG. 7A depicts representative imaging of an RNA-positive control with gene N. FIG. 7B depicts representative imaging of an NTC negative control. RNA template was used at a concentration of 10⁴ copies/μL for the RNA-positive control; 10 mM HAuCl₄; 38.8 mM Na₃Ct; LAMP amplicon:HAuCl₄:Na₃Ct 10:12:1 volume ratio. HAuCl₄ and Na₃Ct were added consecutively on to the LAMP reaction tube directly after reaction with digital images taken after 20 minutes of UV exposure documenting the time of Au(III)-DNA complex formation reflected by the color change to purple for the positive RT-LAMP reaction. FIG. 7C depicts representative images of LAMP reaction chips after HAuCl₄ was dissolved and stored for a week at 4° C. before use with reagents added consecutively. HAuCl₄:Na₃Ct 9:1; Gene E primers and 10⁴ copies/μL RNA for positive control; data representative of three experiments. FIG. 7D depicts representative images of LAMP reaction chips after Na₃Ct was stored for 24 hours at 4° C. before use with reagents added consecutively. HAuCl₄:Na₃Ct 9:1; Gene E primers and 10⁴ copies/μL RNA for positive control; data representative of three experiments.

FIG. 8, comprising FIG. 8A through FIG. 8E, depicts representative data demonstrating the enhanced detection of synthetic SARS-CoV-2 RNA with in-situ gold nanoparticle synthesis for the paper-based colorimetric RT-LAMP. The in-situ gold nanoparticle synthesis method for colorimetric detection of the LAMP amplicons was adopted to enhance the readouts on the paper 5 mm singleplex chips. FIG. 8A depicts representative imaging of studies on the optimization of the HAuCl₄:Na₃Ct volume ratios when reagents are premixed (mixed) then loaded on to the paper or added consecutively (added) directly post reaction. Images were taken with a digital camera every minute during UV exposure by ChemiDoc MP to note the Au(III)-DNA complex formation by change of color to purple. FIG. 8B depicts representative scanned images taken immediately after 5 minutes of UV exposure. FIG. 8C depicts representative scanned images taken 24 hours after 5 minutes of UV exposure, demonstrating stability of the visualization. Gene E primers were used for RNA and NTC samples and 10⁴ copies/μL of the RNA template were used for the RNA and NPC reactions. FIG. 8D depicts a representative graph of mean intensity of purple color for different ratios of HAuCl₄:Na₃Ct for RNA. FIG. 8E depicts a representative graph of mean purple color for different ratios of HAuCl₄:Na₃Ct for NPC. Error bars represent mean±S.D.; n=3.

FIG. 9, comprising FIG. 9A through 9D, depicts schematics and images of a representative paper-based SARS-CoV-2 point-of-care (POC) diagnostic. FIG. 9A depicts a schematic illustrating the process for DNA visualization and enhancement using in-situ formation of gold nanoparticles (AuNPs) combined with RT-LAMP and phenol red. dNTPs: deoxynucleotide triphosphates; A: adenine; T: thymine; G: guanine; C: cytosine. FIG. 9B depicts representative images of multiplex device design optimization with and without resistors and vents. FIG. 9C depicts representative imaging of a pH-based assay on a multiplex device. FIG. 9D depicts representative imaging of enhanced detection using in-situ gold nanoparticle aggregation on a multiplex device.

FIG. 10, comprising FIG. 10A through 10F, depicts a schematic diagram of a representative adhesive bandage for the capture and detection of plasma SARS-CoV-2 IgM and IgG antibodies. FIG. 10A depicts schematics of a representative finger-stick point-of-care device (adhesive bandage). FIG. 10B depicts a schematic of the in-home use of the representative device of FIG. 10A. FIG. 10C depicts a schematic of the valid outcomes of the representative device of FIG. 10A. FIG. 10D depicts schematics of a representative HFA's bioactive layers and working mechanism for gold colloid-based capture and detection of IgM and IgG antibodies from a pinprick (˜15 μL) of blood. Following blood introduction, the plasma (separated from blood cells using a filter membrane) first flows into the hydrophilic conjugate spots, and then into the hydrophilic nitrocellulose detection spots in vertical-lateral-vertical directions (blue arrows). The conjugate spots contain dry AuNP—S and AuNP—N bioconjugates for the capture of IgM and IgG antibodies, together with dry AuNP-IgE complexes to serve as a control. Hydrophilic and hydrophobic regions in the conjugate pad and nitrocellulose membrane are represented in white and grey, respectively. In each spot of the detection zone in the nitrocellulose membrane contains binding surfaces coated with anti-IgM and anti-IgG secondary (capture) antibodies specific to targeted IgM and IgG. In the control spot, anti-IgE antibodies serve to capture only IgE antibodies. FIG. 10E depicts a representative schematic of the back readout of the device when a mixture of IgM and IgG antibodies are introduced to the conjugate pad. FIG. 10F depicts a representative image of a back readout of the HFA showing an active immune outcome (IgM+/IgG+), demonstrating the feasibility of the adhesive bandage. Scale bar: 3 mm.

FIG. 11, comprising FIG. 11A through 11I, depicts representative bioactivation and characterization of AuNPs with SARS-COV-2 S and N proteins. FIG. 11A depicts a schematic of the elemental steps in covalently binding S and N proteins to carboxylated AuNPs via EDC/NHS chemistry. Buffer solutions of pH 7.5 and 11 provide coupling stability for AuNP—S and AuNP—N bioconjugates, respectively. FIG. 11B depicts representative optical density measurements and TEM images (insets) indicating stable (monodispersed) solutions of AuNPs. FIG. 11C depicts representative optical density measurements and TEM images (insets) indicating stable (monodispersed) solutions of AuNPs before and after chemisorption of NHS functional groups to the surface of the AuNP. FIG. 11D depicts representative optical density measurements and TEM images (insets) indicating stable (monodispersed) solutions of AuNPs with NHS before or after chemisorption of S proteins to the activated AuNP surfaces. FIG. 11E depicts representative optical density measurements and TEM images (insets) indicating stable (monodispersed) solutions of AuNPs with NHS before or after chemisorption of N proteins to the activated AuNP surfaces. Scale bars: 200 nm. FIG. 11F depicts representative intrinsic fluorescence measurements of PBS buffer and AuNPs (excitation 240 nm). FIG. 11G depicts representative intrinsic fluorescence measurements of AuNPs and AuNPs functionalized with NHS (excitation 240 nm). FIG. 11H depicts representative intrinsic fluorescence measurements of AuNPs functionalized with NHS and AuNPs functionalized with S protein (excitation 240 nm). FIG. 11I depicts representative intrinsic fluorescence measurements of AuNPs functionalized with NHS and AuNPs functionalized with N protein (excitation 240 nm).

FIG. 12, comprising FIG. 12A through FIG. 12J, depicts representative data from investigations of S and N protein specificity for SARS-CoV-2 IgM and IgG antibodies. FIG. 12A depicts a schematic of the experimental setup, where IgM and IgG antibodies are first physically adsorbed within the hydrophilic detection spots of nitrocellulose membranes, after which AuNP—S solution is directly pipetted onto spots to allow for specific coupling. FIG. 12B depicts the schematic of 12A with AuNP—N solution instead of AuNP—S solution. FIG. 12C depicts a representative image of the front sample side of the assay when AuNP—S solution is added. FIG. 12D depicts a representative image of the front sample side of the assay when AuNP—N solution is added. FIG. 12E depicts a representative image of the back readout side of the assay when AuNP—S solution is added. FIG. 12F depicts a representative image of the back readout side of the assay when AuNP—N solution is added. Scale bars: 3 mm. FIG. 12G depicts quantification of color intensity as in FIGS. 12C and 12E. The inset depicts background images for the front sample and back readout of the assay used as a reference in color intensity quantification. FIG. 12H depicts quantification of color intensity as in FIGS. 12D and 12F. Data presented as mean±SEM; n=6). FIG. 12I depicts representative measurements of intrinsic fluorescence (excitation 240 nm) of AuNP—S binding with or without IgM or IgG antibodies. FIG. 12J depicts representative measurements of intrinsic fluorescence (excitation 240 nm) of AuNP—N binding with or without IgM or IgG. Emission peaks were measured at ˜392 nm (green and red arrows).

FIG. 13, comprising FIG. 13A through 13E, depicts design, characterization, and activation of a representative conjugate pad. FIG. 13A depicts a schematic showing the generation of hydrophobic glass fiber surfaces using PFTS molecules (Step 1), selective removal of PFTS using an acrylic mask, with 3 holes (˜3 mm diameter each) on both sides, and plasma etching (Step 2), activation of hydrophilic spots with AuNP bioconjugates (Step 3), and release of AuNP bioconjugates upon rehydration (Step 4). FIG. 13B depicts representative effects of plasma exposure time on silane desorption values as measured by water contact angle for the front side. FIG. 13C depicts representative effects of plasma exposure time on silane desorption values as measured by water contact angle for the back side. Insets depict representative optical images. Data are presented as mean±S.D.; n=3. FIG. 13D depicts representative imaging of release of AuNP bioconjugates from the hydrophilic conjugate spots upon rehydration. Scale bars: 3 mm. FIG. 13E depicts quantification of the release of AuNP bioconjugates as determined by color intensity. Data are presented as mean±SEM; n=3.

FIG. 14, comprising FIG. 14A through 14D, depicts a representative acrylic mask and characterization of AuNP release upon rehydration. FIG. 14A depicts a representative micrograph of an acrylic mask with a silanized conjugate pad in between its identical front and back parts. The three distinct laser-cut holes (˜3 mm diameter each) on both sides of the mask were used to transfer the spot pattern onto the conjugate pad via plasma etching. Scale bar: 6 mm. FIG. 14B depicts representative optical density measurements of released AuNP—S and AuNP—N bioconjugates from undried (wet, control) and dried conjugate spots upon buffer exposure. FIG. 14C depicts a representative micrograph of released AuNP—S and AuNP—N bioconjugates from undried (wet, control) and dried conjugate spots upon buffer exposure. FIG. 14D depicts representative SEM images of AuNPs before and after release from conjugate spots. Scale bars: 1 μm.

FIG. 15, comprising FIG. 15A through 15F, depicts representative results of sensitivity analysis of an HFA. FIG. 15A depicts a schematic of a representative experimental setup for AuNP—S. FIG. 15B depicts a schematic of a representative experimental setup for AuNP—N. Conjugate pads and nitrocellulose membranes are first activated with 3×3 spots of dry AuNP—SN bioconjugates and anti-IgM and anti-IgG antibodies, respectively. Varying concentrations of IgM and IgG antibodies are then directly pipetted onto conjugate spots and allowed to interact with AuNP bioconjugates. This is followed by gently pressing the conjugate spots to allow antibody-AuNP complexes to interact with capture antibodies in the nitrocellulose spots. FIG. 15C depicts representative images of the results of 15A and controls. FIG. 15D depicts representative images of the results of 15B and controls. Scale bars: 3 mm. FIG. 15E depicts representative quantification of color intensity of assay results as depicted in FIG. 15C. FIG. 15F depicts representative quantification of color intensity of assay results as depicted in FIG. 15D. Data are presented as mean±SEM; n=3.

FIG. 16, comprising FIG. 16A through 16C, depicts representative sensitivity of IgM and IgG antibodies directly allowed to couple with AuNP—S and AuNP—N bioconjugates in their respective buffers. FIG. 16A depicts a schematic representation of an experimental setup, where resulting antibody-AuNP complexes were pipetted directly onto nitrocellulose detection spots activated with anti-IgM and anti-IgG antibodies. FIG. 16B depicts a representative image of results from a sensitivity experiment as depicted in FIG. 16A. Scale bar: 3 mm. FIG. 16C depicts representative overall quantification of color intensity for the four experimental setups in FIG. 16A. Reductions in intensity relative to FIG. 15 are attributed to manual pipetting of solutions onto spots. Data presented as mean±SEM; n=3.

FIG. 17, comprising FIG. 17A through 17E, depicts representative function and long-term storage stability of an example HFA. FIG. 17A depicts a representative experimental setup where conjugate spots, activated with AuNP-IgE (control), AuNP—S, and AuNP—N bioconjugates are aligned with underlying detection spots, activated with anti-IgE (control), anti-IgM, and anti-IgG capture antibodies, for efficient function of the HFA. The background spots used as reference in color intensity quantification are also presented. Prior to experiments, the plasma separation membrane is placed on top of the conjugate pad. FIG. 17B depicts representative images of the four different valid antibody capture and detection conditions, which were tested by pipetting antibody solutions directly onto the plasma separation membrane and gently pressing it. FIG. 17C depicts representative images of positive results after the device was stored for two weeks prior to use. FIG. 17D depicts representative quantification of the color intensity of assay detection zones from experiments as performed in FIGS. 17B and 17C. Data are presented as mean±SEM; n=3. FIG. 17E depicts a representative image of the layers of an HFA combined onto a commercially available adhesive bandage, mimicking the respective schematic of FIG. 10A. Scale bar: 3 mm.

### DETAILED DESCRIPTION

The present invention provides microfluidic porous substrate-based devices for multiplexed biosensing. The devices are suitable for detecting viruses and bacteria, such as by way of detecting pathogenic genes and antibodies. The devices support reverse transcriptase loop-mediated isothermal amplification for rapid results within minutes. The devices also support lateral-vertical-lateral direction flow assays, such as in the form of a multi-layered adhesive bandage.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Porous Substrate-Based Diagnostic Device

The present invention provides porous substrate-based diagnostic devices and methods of use thereof. The devices are cost-effective and easy to use for rapid detection of viruses and bacteria, fragments thereof, and/or antibodies directed against viral or bacterial proteins.

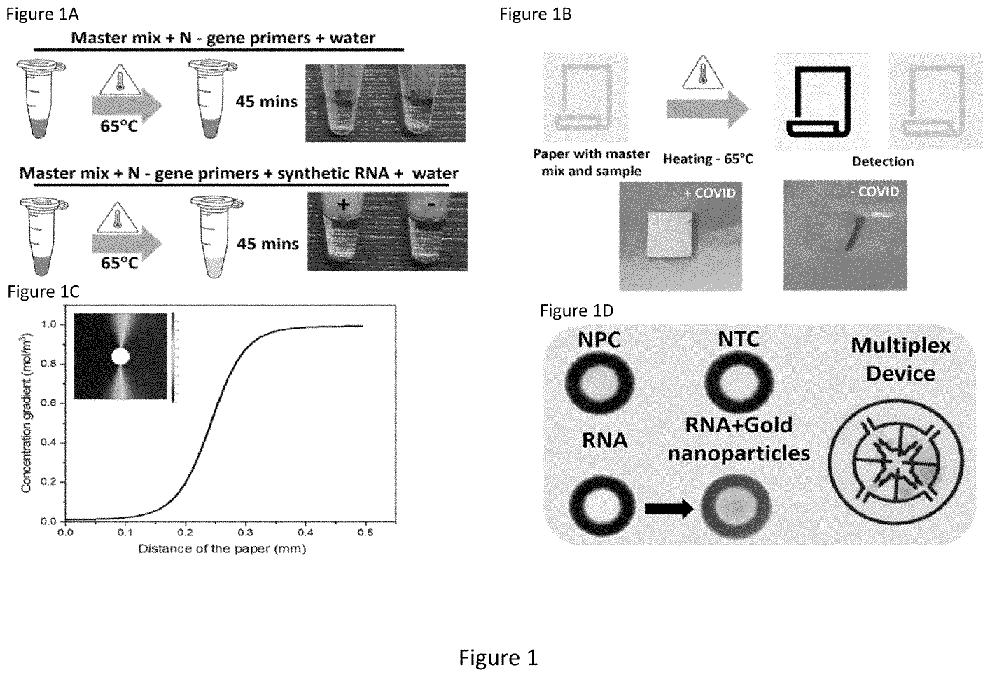

Referring now to FIG. 1D, an exemplary porous substrate-based diagnostic device is depicted. The device comprises a sheet of porous substrate having a pattern of hydrophobic material (black lines) attached thereto. The hydrophobic material permeates through the thickness of the sheet of porous substrate, thereby dividing the sheet into hydrophilic regions fluidly separated by hydrophobic barriers. Contemplated porous substrates include but are not limited to paper, cloth, woven fabrics, non-woven fabrics, threads, yarns, or any other porous materials or perforated films including acrylamide polymers or polyvinyl alcohol, and the like. Contemplated hydrophobic materials include but are not limited to wax, polycaprolactone, poly(ethylene adipate), poly(tetramethylene oxide), poly(trans-butadiene), and other polymers having a melting temperature lower than an ignition or melting temperature of a porous substrate. In various embodiments, a device can comprise one or more discrete microfluidic testing regions, wherein each testing region is fluidly separated by a hydrophobic border. Each testing region comprises a sample spot configured to receive an amount of liquid sample. Contemplated liquid samples include but are not limited to: blood, serum, plasma, saliva, urine, stool, sweat, lacrimal fluid, mucus, and the like.

One or more discrete test chambers may in some embodiments be fluidly connected to the sample spot while being separated from each other by a hydrophobic barrier. The test chambers can be arranged in any desired pattern, such as a radial pattern around a central sample spot, laterally with a sample spot or in any other configuration that allows flow of the sample into one or more test chamber. In some embodiments, each test chamber can be further connected to a hydrophilic region configured to receive excess liquid. In some embodiments, the device of the invention comprises at least 1, 2, 3, 4, 5, 6, 7, 8, 9, 10, 11, 12, 13, 14, 15, 16, 17, 18, 19, 20 or more than 20 test chambers.

Each test chamber can comprise one or more sensing mechanisms commonly used in art, including but not limited to chemically active regions, electrochemical sensors, immobilized capture molecules, probes, and the like. Contemplated probes or capture agents can be any suitable molecule, including antibodies, antibody fragments, antigens, aptamers, bacteriophages, proteins, nucleic acids, oligonucleotides, peptides, lipids, lectins, inhibitors, activators, ligands, hormones, cytokines, sugars, amino acids, fatty acids, phenols, alkaloids, and the like. The probes or capture agents can be configured to capture and thereby detect the presence of any desired molecule in a sample, including proteins, amines, peptides, antigens, antibodies, nucleic acids, steroids, eicosanoids, DNA sequences, RNA sequences, bacteria, viruses, and fragments thereof.

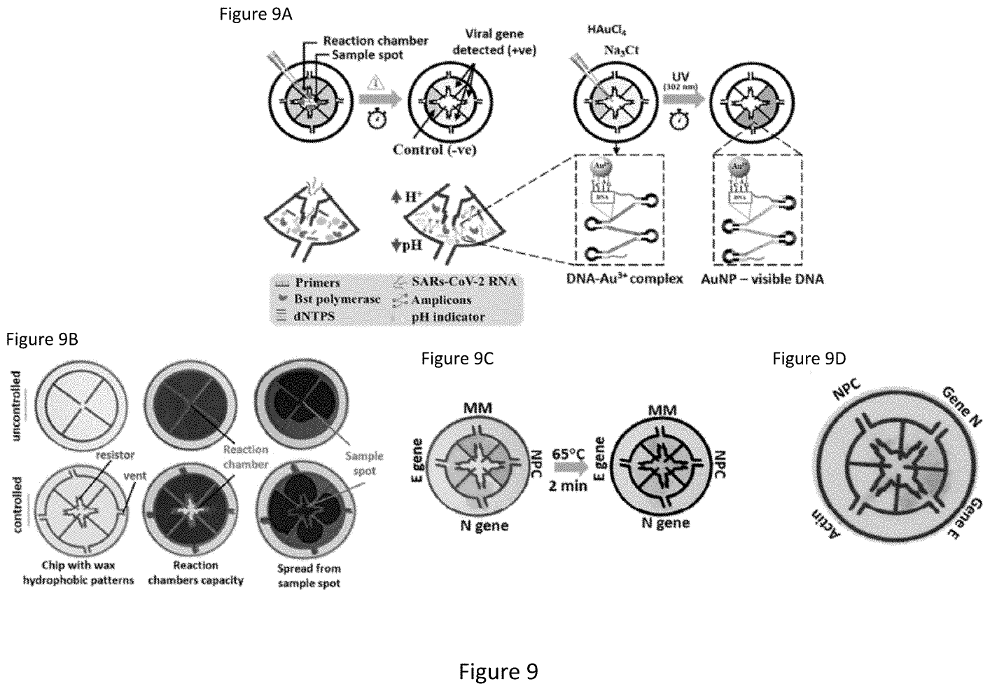

For example, in certain embodiments each test chamber comprises a capture agent to detect a target nucleic acid molecule. For example, in one embodiment, each test chamber comprises primers and LAMP reagents to amplify and detect a target nucleic acid molecule. For example, in one embodiment, each test chamber comprises one or more primers, each primer being specific to a nucleic acid molecule of interest (e.g., a SARS-CoV-2 gene or RNA molecule), one or more polymerases, NTPs or dNTPs, and/or pH indicators (see FIG. 9A, lower left quadrant). In one embodiment, the device comprises a plurality of test chambers, wherein each test chamber comprises a probe or reagent specific to a different target molecule (see e.g., FIGS. 9C and 9D). In one embodiment, the device comprises a control chamber comprising a probe or reagent specific for a molecule known to be present (positive control) or known to be absent (negative control) in a sample. In one exemplary embodiment, the device comprises multiple test chambers, wherein a first test chamber comprises a probe or reagent specific for SARS CoV-2 E, a second test chamber comprises a probe or reagent specific for SARS CoV-2 N, a third test chamber comprises a probe or reagent specific for SARS CoV-2 S, and a fourth test chamber comprises a probe or reagent specific for SARS CoV-2 ORF.

In one embodiment, the one or more hydrophilic test region is loaded with at least one functional particle to enhance the detection of one or more target molecules. In various embodiments, the functional particle is a nanoparticle or microparticle. Examples of nanoparticles include, but are not limited to, gold nanoparticles (AuNPs), silver nanoparticles (AgNPs), platinum nanoparticles (PtNPs), and polymeric nanoparticles. In one embodiment, the functional particle is a quantum dot. Examples of quantum dots include silicon quantum dots, germanium quantum dots, lead quantum dots, cadmium quantum dots, indium quantum dots, zinc quantum dots, gallium quantum dots, and other semiconductor quantum dots. In one embodiment, the functional particle comprises nanowires. In one embodiment, the functional particles are prepared in-situ. In one embodiment, the functional particles are 10-2000 nm in diameter. In one embodiment, the functional particles are 10-500 nm in diameter.

In one embodiment, presence of a target analyte leads to aggregation of the functional particles. In one embodiment, aggregation of the functional particles leads to increased or altered color. Therefore, in some embodiments, the device includes functional particles for amplification of the detectable signal.

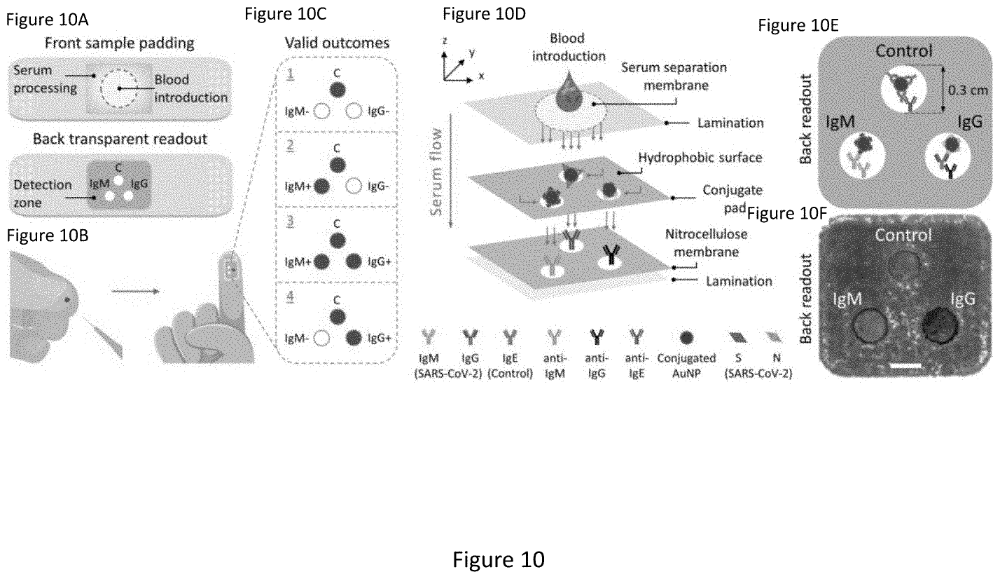

Referring now to FIGS. 10A-10E, another exemplary porous substrate-based diagnostic device is depicted, referred to herein as a hybrid flow assay (HFA) device. The depicted device comprises a plurality of stacked porous layers, wherein one or more porous layers comprises a hydrophobic material attached thereto to support a vertical flow assay. As described above, the hydrophobic material permeates through the thickness of an individual porous layer, thereby dividing the layer into hydrophilic regions fluidly separated by hydrophobic barriers. In various embodiments, the device comprises at least a first porous layer comprising a filter membrane configured to receive a fluid sample, wherein the fluid sample is filtered to permit only desired sample elements to pass through to subsequent porous layers. In one embodiment the thickness of the filter membrane is 0.1 mm to 1 mm.

One or more second porous layers adjacent to the first layer can comprise one or more hydrophilic regions each comprising a probe or capture agent as described above. Liquid samples may flow in lateral directions between each layer when sandwiched between hydrophobic materials. In one embodiment, the liquid sample flows laterally between hydrophobic regions of adjacent sheets of porous substrates. In some embodiments, substrates that can be used for the adjacent sheets include, but are not limited to, glass fibers, polyester fibers, nitrocellulose fibers, polyvinylidene difluoride or any perforated films.

Liquid samples may flow in vertical directions across layers when encountering hydrophilic regions. In this manner, a stacked plurality of porous layers provides a vertical flow assay structure that avoids false negative results typical of the Hook effect. In some embodiments, the liquid sample flows vertically through one or more hydrophilic regions across a sheet of porous substrate made out of nitrocellulose fibers or any other transfer membrane with chemical resistance and mechanical strengths relative to nitrocellulose (e.g., polyvinylidene difluoride), or any perforated films.

The depicted device is in the form of an adhesive bandage, wherein a sample-receiving first layer is configured to contact a wound of a subject and a transparent backing faces outwards to provide a sample readout. However, it should be understood that the device can take any desired form.

In some embodiments, the first sheet comprises at least one biodegradable porous microneedle for finger pricking via in-situ puncturing. In some embodiments, the first sheet comprises at least 2, 3, 4, 5, 6, 7, 8, 9, 10, 11, 12, 13, 14, 15, 16, 17, 18, 19, 20 or more than 20 biodegradable porous microneedles for finger pricking via in-situ puncturing.

In certain embodiments, hydrophilic regions of one or more second porous layers each comprise one or more secondary antibodies configured to bind specifically to an antibody-antigen complex or configured to bind generally to an antibody to be detected. For example, in one embodiment, the secondary antibody is specific for binding to an IgM or IgG antibody region.

In one embodiment, one or more of the hydrophilic regions of one or more porous layers comprises at least one control molecule for validation one or more result from one or more hydrophilic test region. In one embodiment, the control molecule comprises an antibody against IgE.

In one embodiment of a device as depicted in FIGS. 10A-10D, the filter membrane is a plasma separation membrane, which retains blood cells while allowing plasma to flow vertically into the one or more second porous layers adjacent to the membrane. In one embodiment, the filter membrane is a serum separation membrane, wherein blood is captured while allowing serum to flow vertically into the one or more second porous layers adjacent to the membrane. In one embodiment, the one or more second porous layers comprise a conjugate pad. Said conjugate pad can further comprise one or more hydrophilic areas surrounded by hydrophobic barriers. The one or more hydrophilic areas of the conjugate pad can comprise one or more probes or capture agents. In one embodiment, the one or more probes or capture agents can be one or more probes or capture agents. Example probes and capture agents include, but are not limited to, antibodies, antibody fragments, antigens, aptamers, bacteriophages, proteins, nucleic acids, oligonucleotides, peptides, lipids, lectins, inhibitors, activators, ligands, hormones, cytokines, sugars, amino acids, fatty acids, phenols, alkaloids, and the like. In some embodiments, the antigens can be used to capture disease-associated antibodies. In some embodiments, the antigens can be used to capture protective antibodies related to a pandemic or outbreak. In some embodiments, the antigens can be used to capture self-antibodies associated with an autoimmune disease or disorder.

In certain embodiments hydrophilic regions of one or more second porous layers each comprise one or more functional particles. In various embodiments, the functional particle is a nanoparticle or microparticle. Examples of nanoparticles include, but are not limited to, gold nanoparticles (AuNPs), silver nanoparticles (AgNPs), platinum nanoparticles (PtNPs), and polymeric nanoparticles. In one embodiment, the functional particle is a quantum dot. Examples of quantum dots include silicon quantum dots, germanium quantum dots, lead quantum dots, cadmium quantum dots, indium quantum dots, zinc quantum dots, gallium quantum dots, and other semiconductor quantum dots. In one embodiment, the functional particle comprises nanowires. In one embodiment, the functional particles are prepared in-situ. In one embodiment, the functional particles range in size from 10-500 nm in diameter.

In various embodiments, the functional particles are conjugated to one or more antigens. Exemplary antigens that can be conjugated to the functional particles include pathogenic and non-pathogenic antigens as are discussed in detail elsewhere herein. In some embodiments, the antigens can be used to capture protective antibodies related to a pandemic or outbreak.

In one embodiment, the one or more nanoparticles are conjugated to one or more test antigens. One or more antibodies specific for the one or more antigens from the test sample can then bind to the one or more antigens conjugated to the nanoparticles and flow vertically from the conjugate pad to one or more third porous layers adjacent to the conjugate pad on the opposing face of the first porous layer.

In one embodiment, the device comprises one or more third porous layers comprising a detection zone. In one embodiment the one or more third porous layers are comprised of one or more nitrocellulose membranes. In one embodiment, the detection zone comprises one or more hydrophilic spots surrounded by hydrophobic barriers. In one embodiment, the hydrophilic spots of the detection zone are aligned with the hydrophilic spots of the conjugate pad. In certain embodiments, the one or more hydrophilic spots of the detection zone comprise one or more capture and/or reporter agents. In one embodiment, the one or more capture and/or reporter agents comprises secondary antibodies which specifically bind the one or more antibodies bound to the one or more antigens conjugated to the nanoparticles from the conjugate layer. In one embodiment, the detection zone comprises one or more hydrophilic spots which comprise one or more capture and/or reporter agents that act as a control for assay result validity.

In one embodiment, the detection zone is in contact with a transparent impermeable layer opposite the conjugate pad, allowing visualization of the detection zone.

### Multiplex Diagnostic Systems

In one embodiment, one or more device of the invention can be integrated into a multi-component system for detection of one or more target molecule.

In some embodiments, one or more paper-based LAMP diagnostic device of the invention can be integrated into a system comprising one or more additional diagnostic device. In some embodiments, the one or more additional diagnostic device is one or more additional paper-based LAMP diagnostic device. In some embodiments, the one or more additional diagnostic device is one or more additional non-paper-based diagnostic device.

In some embodiments, one or more HFA diagnostic device of the invention can be integrated into a system comprising one or more additional diagnostic device. In some embodiments, the one or more additional diagnostic device is one or more additional HFA diagnostic device. In some embodiments, the one or more additional diagnostic device is one or more additional non-HFA diagnostic device.

In some embodiments, one or more paper-based LAMP diagnostic device of the invention can be integrated into a system further comprising one or more HFA device of the invention.

In some embodiments, the multiplex diagnostic system of the invention serves as a diagnostic system for detection two different biomarkers or analytes associated with one or more different diseases, disorders or pathogens. For example, in some embodiments, the system detects one or more nucleic acid molecule associated with one or more diseases, disorders or pathogens and further detects one or more antibody against one or more diseases, disorders or pathogens. In some embodiments, the multiplex diagnostic system of the invention serves as a diagnostic system for detection two different biomarkers or analytes associated with the same disease, disorder or pathogen. For example, in some embodiments, the system detects both a nucleic acid molecule from a specific pathogen and an antibody against the same pathogen.

### Methods

In one embodiment, a device of the invention can be used for diagnostics and other analytical applications, such as to detect an analyte of interest. In one embodiment, reagents are administered to the one or more hydrophilic channels to detect the presence of analytes in a fluid (e.g., a biological sample, an environmental sample, or an industrial sample). In some embodiments, the response to the analyte is visible to the naked eye. For example, to the administered reagents provide a color indicator of the presence of the analyte. Indicators may include molecules that become colored in the presence of the analyte, change color in the presence of the analyte, or emit fluorescence, phosphorescence, or luminescence in the presence of the analyte. In other embodiments, radiological, magnetic, optical, and/or electrical measurements can be used to determine the presence of proteins, antibodies, or other analytes.

In some embodiments, to detect a specific protein, one or more hydrophilic regions can be derivatized with reagents, such as small molecules, that selectively bind to or interact with the analyte. Or, for example, to detect a specific antibody, one or more hydrophilic regions can be derivatized with reagents such as antigens, that selectively bind to or interact with the antibody to be detected. In one embodiment, the hydrophilic regions comprise one or more antibodies, or antibody fragments, that selectively bind a peptide or protein to be detected. In one embodiment, the hydrophilic regions comprises one or more nucleic acid probes, that selectively binds a nucleic acid molecule or sequence to be detected.

Reagents such as small molecules, antibodies, antibody fragments, nucleic acid probes, and/or proteins can be covalently or non-covalently linked to at least one hydrophilic regions, using similar chemistry to that used to immobilize molecules on beads or glass slides, or using chemistry used for linking molecules to carbohydrates. In alternative embodiments, the reagents may be applied and/or immobilized by applying them from solution and allowing the solvent to evaporate. The reagents can be immobilized by physical absorption onto at least one hydrophilic region by other non-covalent interactions. In general, a wide variety of reagents can be used to detect analytes and can be applied by a variety of suitable methods. These reagents could include antibodies, nucleic acids, aptamers, molecularly imprinted polymers, chemical receptors, proteins, peptides, inorganic compounds, and organic small molecules. These reagents could be adsorbed to at least one hydrophilic region (non-covalently through non-specific interactions), or covalently (as either esters, amides, imines, ethers, or through carbon-carbon, carbon-nitrogen, carbon-oxygen, or oxygen-nitrogen bonds).

In one embodiment, one or more hydrophilic regions may further comprise nanoparticles, enzymes, oligonucleotides, etc. to enhance detection capabilities of the device.

In some embodiments, the interaction of some analytes with some reagents results in a colorimetric change that can be detected visually. However, in some embodiments, the interaction of some analytes with some reagents may not result in a visible color change, unless the analyte was previously labeled. In such an embodiment, one or more hydrophilic regions can be additionally treated to add a stain or a labeled protein, antibody, nucleic acid, or other reagent that binds to the target analyte after it binds to the reagent in one or hydrophilic regions and produces a visible color change.

A device of the invention may be used in a number of different applications. For example, it can be useful for at-home diagnostics, pediatric physicians; physicians working in resource-poor settings such as developing countries; physicians working in emergency or point-of-care environments; nurses or caregivers in nursing homes; military technologists; athletes, trainers, or sports physicians/technicians; veterinarians; farmers or agricultural scientists/engineers; environmental scientists; and chemists, bioengineers, or chemical engineers.

In certain aspects, the present disclosure provides methods of performing diagnostics for detection of a target molecule in a liquid sample. Exemplary target molecules that can be detected using a device of the invention include, but are not limited to, nucleic acid molecules including DNA and RNA molecules, antibodies, antigens, small molecules.

In some embodiments, the target molecule is a biomarker of a disease or disorder. In one aspect, the devices of the present invention are useful for detecting or diagnosing a disease or disorder associated with a targeted biomarker. In one embodiment, devices of the invention can be used diagnostically to monitor the presence of one or more biomarker in a sample as part of a clinical testing procedure, e.g., to determine the efficacy of a given treatment regimen.

In some embodiments, the methods of the invention include performing an assay using a device of the invention. In one embodiment, the invention includes the use of a device of the invention in any bioassay that can be used to determine the presence of at least one target molecule.

In certain embodiments, a device of the invention may be used to detect a biomarker from a pathogenic or potentially pathogenic microbe, including a food borne pathogen, or a water borne pathogen. In one embodiment, the pathogen is pathogenic to humans. In one embodiment, the pathogen is pathogenic to non-humans (e.g., a non-human mammal pathogen, a plant pathogen, a marine animal pathogen or an insect pathogen.) A pathogenic microbe can be a virus, a bacterium, and/or a fungus. In certain aspects, a device of the invention can be configured to detect a variety of microbes including viruses, bacteria, and fungi simultaneously.

In certain aspects, a microbe includes a virus. The virus can be from the Adenoviridae, Coronaviridae, Filoviridae, Flaviviridae, Hepadnaviridae, Herpesviridae, Orthomyxoviridae, Paramyxovirinae, Pneumovirinae, Picornaviridae, Poxyiridae, Retroviridae, or Togaviridae family of viruses; and/or Parainfluenza, Influenza, H5N1, Marburg, Ebola, Severe acute respiratory syndrome coronavirus, Yellow fever virus, Human respiratory syncytial virus, Hantavirus, or Vaccinia virus. In some embodiments, the virus is Severe acute respiratory syndrome coronavirus 2 (SARS-CoV-2), smallpox, influenza, mumps, measles, chickenpox, Ebola, HIV, or rubella.

In yet another aspect, the pathogenic or potentially pathogenic microbe can be a bacteria. A bacterium can be an intracellular, a gram positive, or a gram-negative bacteria. In a further aspect, bacteria include, but is not limited to a Neisseria meningitidis (N. meningitidis), Streptococcus pneumoniae (S. pneumoniae), and Haemophilus influenzae type B (Hib), B. pertussis, B. parapertussis, B. holmesii, Escherichia, a Staphylococcus, a Bacillus, a Francisella, or a Yersinia bacteria. In still a further aspect, the bacteria is Bacillus anthracis, Yersinia pestis, Francisella tularensis, Pseudomonas aerugenosa, or Staphylococcus aureas. In still a further aspect, a bacteria is a drug resistant bacteria, such as a multiple drug resistant Staphylococcus aureas (MRSA). Representative medically relevant Gram-negative bacilli include Hemophilus influenzae, Klebsiella pneumoniae, Legionella pneumophila, Pseudomonas aeruginosa, Escherichia coli, Proteus mirabilis, Enterobacter cloacae, Serratia marcescens, Helicobacter pylori, Salmonella enteritidis, and Salmonella typhi. Representative gram-positive bacteria include, but are not limited to Bacillus, Listeria, Staphylococcus, Streptococcus, Enterococcus, Actinobacteria and Clostridium mycoplasma that lack cell walls and cannot be Gram stained, including those bacteria that are derived from such forms.

In still another aspect, the pathogenic or potentially pathogenic microbe is a fungus, such as members of the family Aspergillus, Candida, Crytpococus, Histoplasma, Coccidioides, Blastomyces, Pneumocystis, or Zygomyces. In still further embodiments a fungus includes, but is not limited to Aspergillus fumigatus, Candida albicans, Cryptococcus neoformans, Histoplasma capsulatum, Coccidioides immitis, or Pneumocystis carinii. The family zygomycetes includes Basidiobolales (Basidiobolaceae), Dimargaritales (Dimargaritaceae), Endogonales (Endogonaceae), Entomophthorales (Ancylistaceae, Completoriaceae, Entomophthoraceae, Meristacraceae, Neozygitaceae), Kickxellales (Kickxellaceae), Mortierellales (Mortierellaceae), Mucorales, and Zoopagales.

In one embodiment, a device of the invention may be used for drug testing and receptor binding assays. In one embodiment, a device of the invention may be used for biosensing or chemo sensing of the biological molecules such as blood glucose. In one embodiment, a device of the invention may be used for low-cost biosensing applications for the detection of disease specific biomarkers such as lactate, uric acid, glucose ions and/or salt concentrations. In one embodiment, a device of the invention may be used for the detection of infectious diseases such as COVID-19, seasonal flu, tuberculosis, typhoid, dengue, malaria etc. In one embodiment, a device of the invention may be used for detection of and non-communicable diseases including but not limited to obesity, diabetes, cardiovascular disorders, hypertension, cancer, etc. by detecting specific biomarkers in the body fluids (e.g., blood, urine, tears, saliva). In one embodiment, a device of the invention may be used for allergen detection. In one embodiment, a device of the invention can be used for analyzing histamine release from immune cells.

In certain embodiments a device of the invention may be configured for diagnosis in a laboratory or home setting. In other embodiments a device of the invention may be configured to provide a point of care device for home or field diagnosis. Furthermore, the device and method presented may be used to detect various plant, animal, food-borne, and other infectious diseases or non-infectious diseases in resource-limited settings.

In some embodiments, one or more target molecules or analytes is detected in a liquid sample. In some embodiments, the liquid sample is a liquid biological sample. Example liquid biological samples include, but are not limited to, blood, plasma, serum, saliva, sputum, plasma, urine, sweat, stool, lacrimal fluid, bronchoalveolar lavage fluid, cerebrospinal fluid, mucus, breast milk, tissue extract and the like.

In one embodiment, the device of the invention may be used for assaying small volumes of biological samples, e.g., fluid samples. In some embodiments, a single drop of liquid, e.g., a drop of blood from a pinpricked finger, is sufficient to perform assays providing a simple yes/no answer to the presence of a target molecule or analyte, or a semi-quantitative measurement of the amount of analyte that is present in the sample, e.g., by performing a visual or digital comparison of the intensity of the assay to a calibrated color chart.

In one embodiment, the disclosure provides methods of detecting one or more target nucleic acid molecules. In one embodiment, the method of detecting one or more target nucleic acid molecules comprises the steps of:

a) collecting a sample from a subject and/or patient;

b) introducing the sample to the receiving or sample area of a device of the present disclosure; and

c) interpreting the results displayed in the one or more test chambers and/or detection zones.

In some embodiments, the device is loaded with at least one set of primers specific for the target nucleic acid molecule and pH sensitive reagents for a colorimetric LAMP assay. In some embodiments, the method further comprises the step of b1) heating the device in between steps b) and c). In some embodiments the step of b1) comprises heating the device to a temperature sufficient for performing the colorimetric LAMP assay for a period of time sufficient to produce a pH change that can be detected by a visual colorimetric change. In some embodiments, the device is heated to a temperature in the range of 50-70° C. for a duration of at least 30 minutes. In some embodiments, the device is heated to a temperature in the range of 60-65° C. for a duration of 40-60 minutes. In some embodiments, the method further comprises the step b2) of adding a reporting agent between steps b) and c). In one embodiment, the method further comprises the steps of b) adding a reporting agent; and b3) exposing the device to ultraviolet light after step b) and before step c). In one embodiment, the method further comprises the step of b1) heating the device; b2) adding a reporting agent; and b3) exposing the device to ultraviolet light after step b) and before step c).

In one embodiment, step c) further comprises the step of determining the validity of the test results by examining the results of the control region of the device, wherein the results are valid if the control region presents a positive result.

In one embodiment, the disclosure provides methods of detecting one or more antibodies against one or more test antigens. In one embodiment, the method of detecting one or more antibodies against one or more test antigens comprises the steps of:

a) collecting a sample from a subject and/or patient;

b) introducing the sample to the receiving or sample area of a device of the present disclosure; and

c) interpreting the results displayed in the one or more test chambers and/or detection zones.

In some embodiments, the device is loaded with at least one test antigen. In one embodiment, the device is loaded with at least one test antigen and at least one control binding molecule. In one embodiment, the control binding molecule is an anti-IgE antibody.

In some embodiments, the device is loaded with at least one secondary antibody specific to the isotype of the targeted antibody. For example, in one embodiment, the device is loaded with at least one anti-IgM or anti-IgG secondary antibody. In some embodiments, the control region of the device is loaded with anti-IgE antibodies which serve to capture IgE antibodies.

In one embodiment, step c) further comprises the step of determining the validity of the test results by examining the results of the control region of the device, wherein the results are valid if the control region presents a positive result.

In one embodiment, the method comprises the steps of:

a) pricking the skin of a subject and/or patient such that a drop of blood is exuded from the pinprick;

b) applying a device of the present disclosure to the skin of the subject and/or patient such that the drop of blood enters the receiving or sample area of the device; and

c) interpreting the results displayed in the one or more test chambers and/or detection zones.

In one embodiment, step c) further comprises the step of determining the validity of the test results by examining the results of the control region of the device, wherein the results are valid if the control region presents a positive result (see FIGS. 10A-10C).

In one embodiment, step c) further comprises the step of determining a positive test result by examining the result of the test region of the device, wherein the results is positive based on detection of a target antibody bound to a test antigen in the test region (see FIGS. 10A-10C).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, utilize the present invention and practice the claimed methods. The following working examples, therefore, specifically point out exemplary embodiments of the present invention, and are not to be construed as limiting in any way the remainder of the disclosure.

### Example 1: Paper-Based LAMP Diagnostic Device

The present study relates to an affordable, rapid paper-based multiplex test for the detection of pathogens by Loop Mediated Isothermal Amplification (LAMP). LAMP is a tube-based diagnostic test that amplifies genetic materials and provides results between 30-60 minutes. In the present study, a paper-based LAMP device is developed for multiplex detection, which could be a key player in current and future epidemics/pandemics. Here, the paper-based LAMP technology is applied for multiple SARS-CoV-2 genes, but the technology can be extended to other viral and bacterial diagnostics. The device consists of a paper chip divided into distinct regions (gene detection and control regions) and linked to a sample loading spot (FIG. 1). Primers and other reagents are preloaded to the device before freezing or dry storage. Devices are then retrieved on demand, where the clinical sample is loaded on the device's sample spot and flows homogenously to the assay/analysis regions. The assay will get initiated upon heating the device on a hotplate, then results can be detected visually by color change of the genes regions. A smartphone camera can also be used for a medical data record. The device is portable and easy to use, making it applicable for home use, remote areas, and other places.

Validation and Optimization of LAMP Assay with Custom Primers

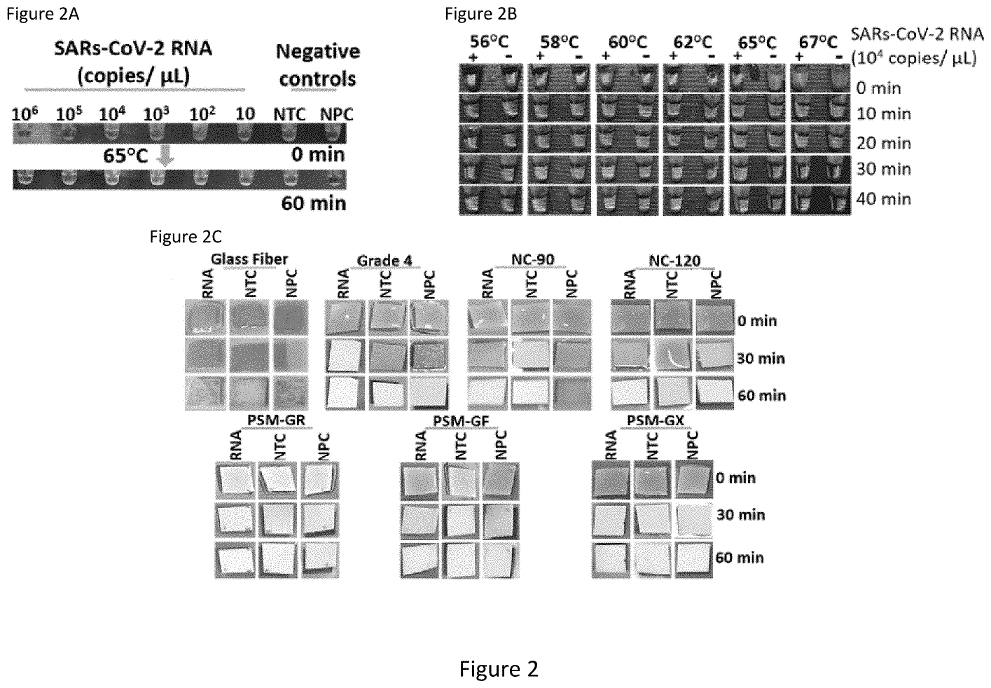

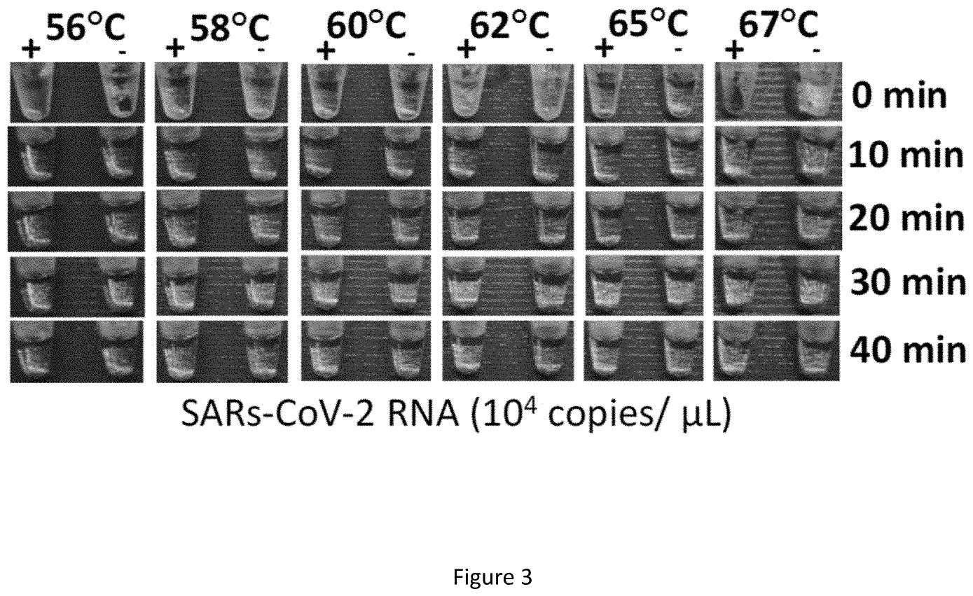

To validate the LAMP assay, conventional tube-based RT-LAMP was carried out using a synthetic SARS-CoV-2 RNA and a colorimetric readout obtained (FIG. 1A). Two sets of primers were evaluated for the assay, one targeting the SARS-CoV-2 N gene, and the other targeting the E gene. As depicted in FIG. 1A, gene N was tested with the recommended RT-LAMP assay temperature (Tm) of 65° C. in a heating block with 300 rpm shaking for 40 minutes, after which the positive RNA-containing reaction turned yellow, and the negative No Template Control (NTC) remained pink (FIG. 1B). Upon being added to paper, more molecules were concentrated in the middle region indicated by darker color in the middle than on the sides of the paper. COMSOL simulation was performed to examine the diffusion of reagents on porous filter paper and indicated a short-range gradient (FIG. 1C). To confine the reagents on paper, printed wax microspots were fabricate, laminated at the bottom, and the pH-based LAMP reagents were added and heated. Control (NTC and No Primer Control (NPC)) remained pink whereas the RNA sample turned yellow indicating COVID-positive sample. As the yellow color on paper is pale, gold (III) chloride trihydrate (HAuCl₄.3H₂O) and trisodium dihydrate citrate (Na₃Ct.H₃O), which turn purple upon UV-treatment, were added to enhance detection color. Upon addition of gold (III) chloride trihydrate and trisodium dihydrate citrate and UV treatment samples with RNA for the E and N genes turned purple whereas the master mix-only control and NPC turned white in color (FIG. 1D). To optimize the conventional assay the analytical sensitivity was tested for the limit of detection (LOD) with synthetic SARS-CoV-2 RNA titrated from 1 million viral copies (or virus particles)/μL (10⁶) down to a single copy using serial log-10 dilutions (FIG. 2A). 25 μL reactions were incubated at 65° C. in an oven and readouts taken at 0 and 60 minutes. As shown, the LoD for the E gene was 10² copies/μL of synthetic RNA. The assay was then optimized for Tm for the utilized primers and the heating time. 25 μL reaction volumes were incubated at a range of temperatures from 56° C. to 67° C. and monitored qualitatively every 10 minutes for the E gene (FIG. 2B). Optimal assay conditions would yield solid resolution (easy distinction of positive from NTC) and fast results (short Time-to-Results (TtR)) with minimal background. As depicted in FIGS. 2B and 3, 60° C. and 62° C. were found to be optimal for the E and N genes respectively.

### Membrane Compatibility and Selection

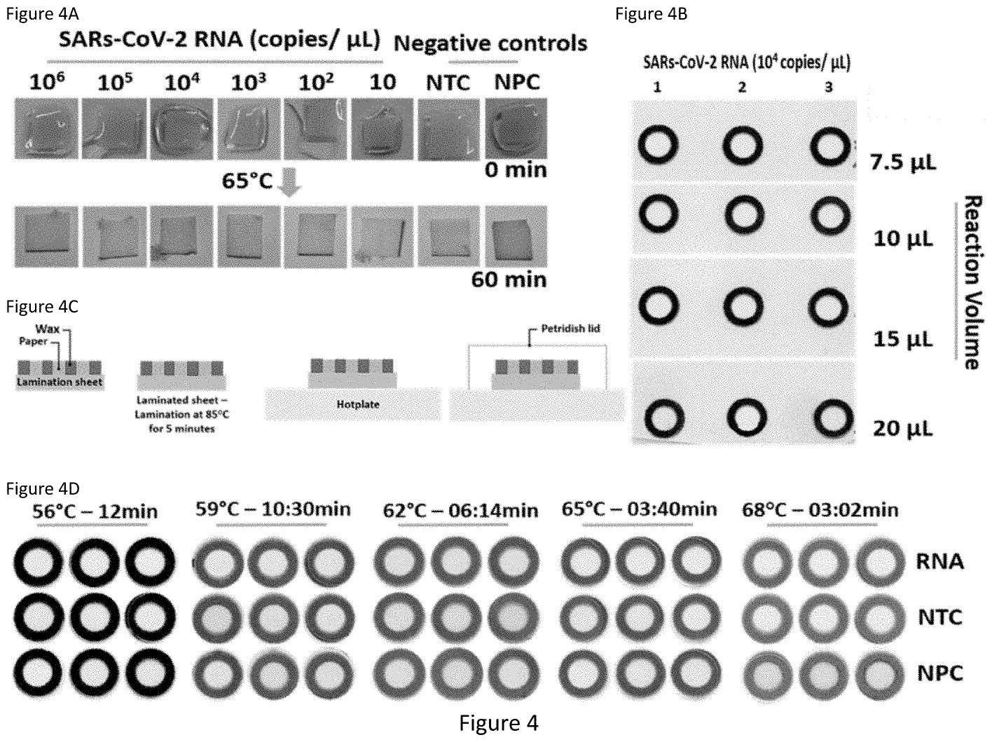

To determine the appropriate reaction membrane for a paper-based RT-LAMP device, various materials were screened by performing RT-LAMP reactions on the materials. As seen in FIG. 4, the ˜5 mm×5 mm pads were loaded with 25 μL premixed RT-LAMP reaction solution. Three types of reactions were performed for each membrane: a template positive (RNA—10⁶ copies/μL), an NTC (0 copies/μL), and an NPC (RNA—10⁶ copies/μL, no primer). The reactions were incubated at 65° C. for 1 hour in sterile and sealed petri dishes to limit evaporation, with readouts taken at 0, 30, and 60 minutes. Grade 4 cellulose showed results closest to those expected from the conventional in-solution RT-LAMP reactions and had the shortest TtR among the tested materials (FIG. 2C), although nitrocellulose (NC-90) appeared as a potential alternative, albeit with a longer TtR and yellowing of the NTC. Grade 4 Whatman™ cellulose filter paper has moderate pore size, is biocompatible with nucleic acid amplification, and does not inhibit reagent activities or interfere with phenol red reaction unbiased results (Kaarj, K., et al., 2018, Scientific Reports, 8(1):1-1). S14 glass fiber appeared to inhibit the reaction or provide alkaline reaction conditions which prevented the pH detection of the assay by phenol red. A similar pH bias to acidic conditions was observed with PSM-GR. PSM-GF and PSM-GX, composed of hydrophilic polyethersulfone fibers, appeared to inhibit the RT-LAMP reaction.

### Parameter Optimization for Single Target Paper-Based RT-LAMP Detection of SARS-CoV-2

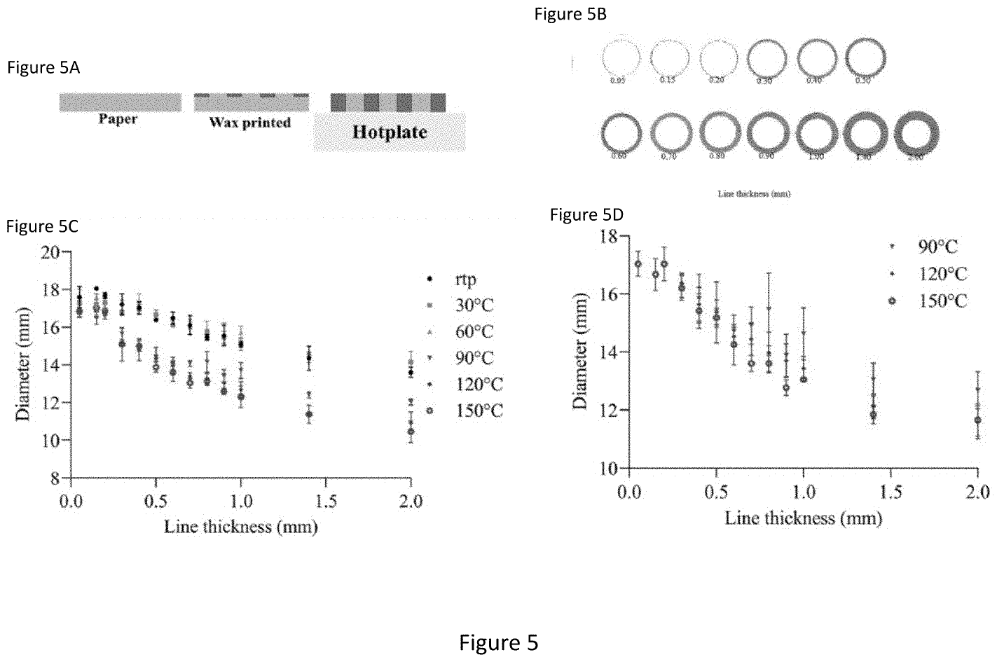

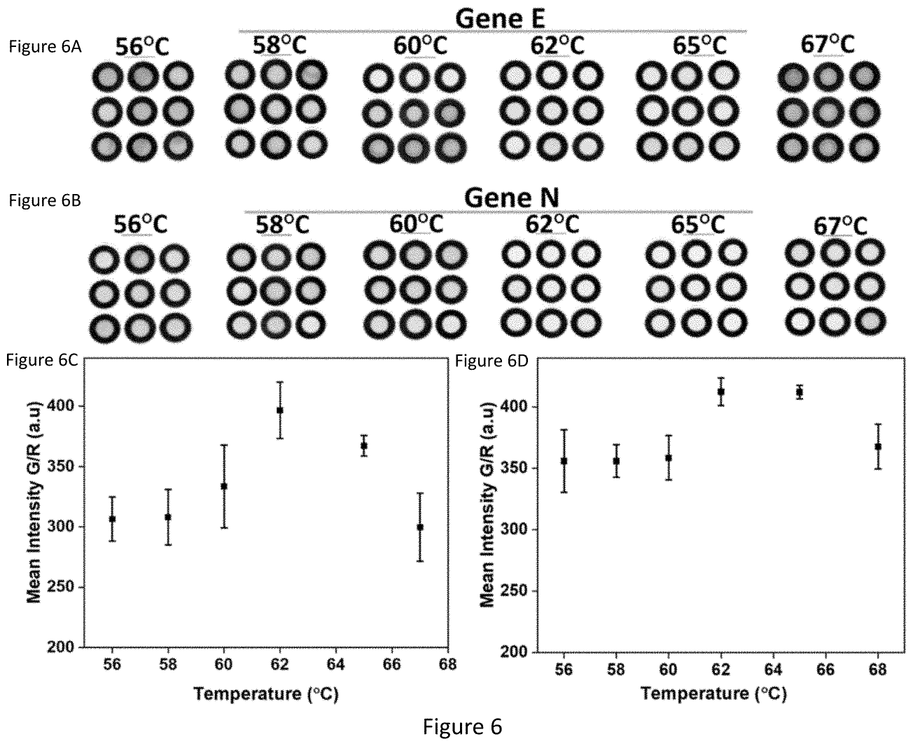

As optimal RT-LAMP parameters may differ from in-solution assays when performed on a 2D matrix, assay optimization was repeated for grade 4 cellulose filter paper. FIG. 4A depicts the 10 copies/4, LoD for the E gene of SARS-CoV-2, which was determined by premixing the reaction components then loading 25 μL on the 5 mm×5 mm grade 4 cellulose pads and incubation at 65° C. for one hour in sealed petri dishes. To confine the reaction membranes, printed wax spots 5 mm in diameter were fabricated using a Xerox ColorQube 8570DN, resulting in reaction spots that act as singleplex chips. The hydrophobic barrier was optimized based on line thickness and wax transfer heating temperature at a fixed time (FIG. 5). A line thickness of 0.9 mm at a temperature of 120° C. for 3 minutes was found to be optimal to form the hydrophobic barrier. The optimal reaction volume the 5 mm spot can hold was determined as seen in FIG. 4B, in which different positive N gene reaction volumes were loaded in triplicate and incubated at 65° C. A reaction volume of 7.5 μL was found to yield readable results without leaking and future reactions were performed with 8 μL reaction volume per singleplex chip. A schematic of the workflow of laminating the chip bottoms and performing the assay is depicted in FIG. 4C. For the N gene (FIG. 4D) the shortest TtR was 3 minutes for a reaction at 68° C., thus Tm optimization assays were performed for 4 minutes at a range of temperatures from 56-67° C. with 10⁴ copies/4, RNA template (FIGS. 6A and 6B). As reactions at 60 and 62° C. were optimal for the E gene and 62 and 65° C. were optimal for the N gene, 62° C. was chosen for future assays (FIGS. 6C and 6D).

### Enhanced Visualization on Cellulose Chips

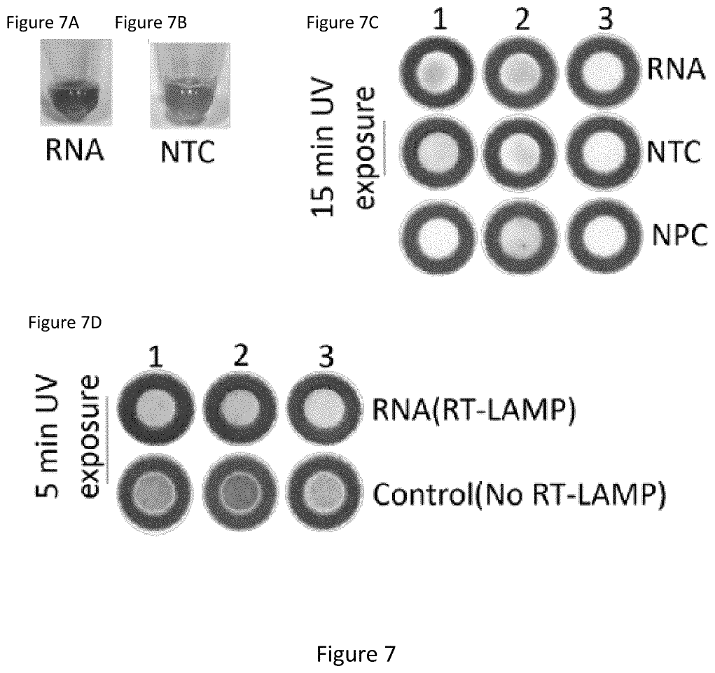

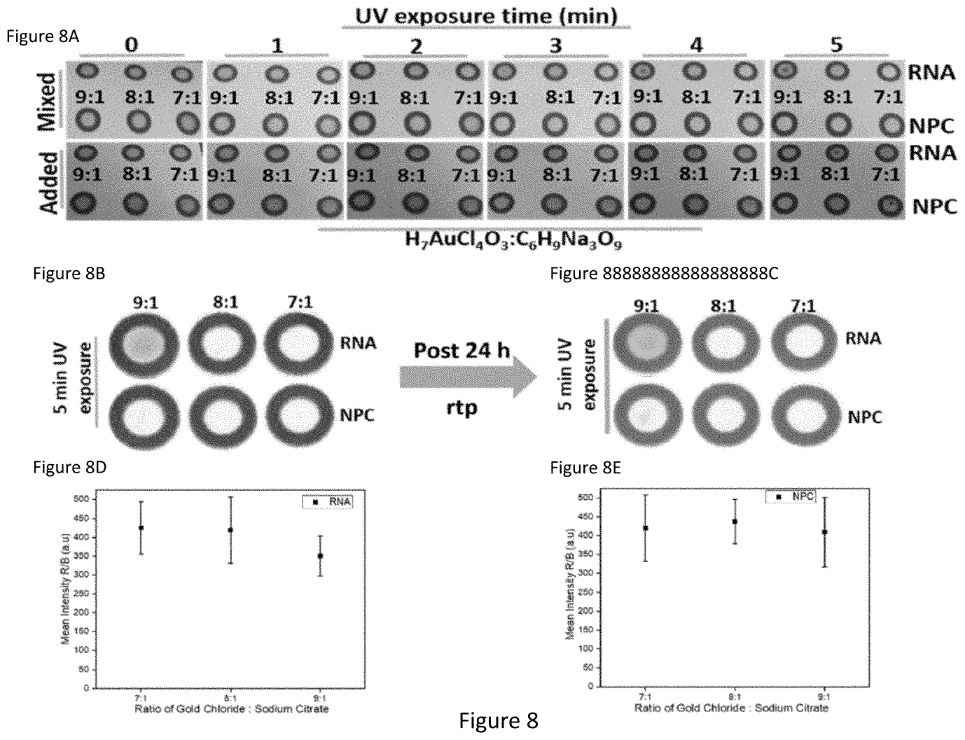

Most results presented were scanned with an Epson Perfection V850 Pro scanner, which improved color intensity. To improve color intensity further, in-situ gold nanoparticle (AuNPs) synthetic methods for colorimetric detection of the LAMP amplicons were examined using gold (III) chloride trihydrate and trisodium citrate dihydrate at a concentration of 1 mM and 38.8 mM respectively (Sivakumar, R., et al., 2021, Lab on a Chip, 21:700-709). The method was initially validated in tube-based reactions, after which HAuCl₄ and Na₃Ct were added to the LAMP amplicons at a volume ratio of 10:12:1 and illuminated with UV light for 20 minutes in a Chemidoc at a wavelength of 203 nm (FIGS. 7A and 7B). Yielding positive results, the experiments were replicated on cellulose chips by either premixing the HAuCl₄ and Na₃Ct in a tube before adding it to the chips post-reaction or adding the reagents sequentially directly to the chip (FIG. 8A). Although HAuCl₄ and Na₃Ct were kept at fixed concentrations, the ratio of HAuCl₄:LAMP amplicons was optimized by varying the volumetric ratios at 9:1, 8:1, and 7:1, with the volume of Na₃Ct kept constant. The ratio of 9:1 displaying a purple color in less than 5 minutes while 8:1 and 7:1 displayed no visible change by 5 minutes of UV exposure. After 5 minutes of UV exposure the chips were incubated overnight at room temperature and examined again to show the stability of the detection method (FIGS. 8B-8E). Additional experiments demonstrated that HAuCl₄ and Na₃Ct must be prepared fresh for optimal results (FIGS. 5C and 5D) The AuNPs aggregation strategy is illustrated in FIG. 9A, an Au (III)-DNA complex is formed by the Au3+ binding to the nitrogenous bases and not the phosphate groups of DNAs (Koo, K. M., et al., 2015, Analytical Methods, 7(17):7042-7054). This Au (III)-DNA complex gets reduced to form AuNPs, upon exposure to UV light or a reducing agent (Sohn, J. S., et al., 2011, Molecules, 16(10):8143-8151; Berti, L., et al., 2005, Journal of the American Chemical Society, 127(32):11216-11217). Here, the presence of Na₃Ct is essential in AuNP synthesis because it acts as a reducing and stabilizing agent in the reaction (Ji, X., et al., 2007, Journal of the American Chemical Society, 129(45):13939-13948). Therefore, if the reaction is positive and LAMP amplicons are present, AuNPs will form in situ under the needed conditions turning from colorless to pinkish purple. This purple color of the synthesized AuNPs is more visible than the pale-yellow obtained with only phenol red.

### Multiplex Device Design

Multiplex devices were designed in AutoCAD and fabricated using a Xerox ColorQube 8570DN. The device was designed, printed, laminated, and optimized with (controlled) and without (uncontrolled) several features (FIG. 9A, including (1) a sample spot that distributes the sample equally to (2) four radial reaction chambers through (3) fluidic resistors that prevent backflow, (4) fluidic ports for venting excess liquid from the reaction chambers, and (5) an outer ring for containing overflowing fluids. Orange dye was loaded to the chips and the controlled chip allowed loading of reagents into the reaction chambers without contamination of the sample spot (FIG. 9B). Blue dye was loaded on the sample spot, which wicked the sample into the reaction chambers within seconds, covering the majority of the chamber's areas.

### RT-LAMP Assay on a Multiplex Device

The four quadrant reaction chambers can be loaded with different reagents for detecting different pathogens, but for demonstration purposes different genes of SARS-CoV-2 were utilized. The wax-printed hydrophobic walls were optimized with 0.3 mm line thickness and the reaction chambers optimized with 35 μL volume. The fluidic resistors were optimized to prevent reagents and sample backflow and quadrant-crosstalk. In fabricating the sample SARS-CoV-2 multiplex device with bottom lamination, reagents of controls (NPC, master mix), N gene, and E gene were loaded into each reaction chamber. 50 μL of total RNA sample (10⁵ copies/μL) at a ratio of 1:49 RNA:water was loaded to the sample spot, which splits equally to the reaction chambers. The device was heated on a hotplate for 2 minutes and the readouts visualized as yellow (positive) and pink (negative) in each quadrant (FIG. 9C). To further enhance the detection on multiplex devices each quadrant consisted of reagents for NPC, actin control, N gene, and E gene with in-situ gold nanoparticle visualization (FIG. 9D).

### Example 2: Adhesive Bandage for SARS-CoV-2 Immune Response Detection and Screening

Since its first occurrence in December 2019, the coronavirus disease 2019 (COVID-19) pandemic has reached, as of Oct. 6, 2022, a grim landmark with more than 616 million confirmed cases worldwide, including 6.5 million deaths. However, the incidence of disease is believed to be underdiagnose and thus underreported. For example, in the United States alone it is estimated that for every reported case there are 3 to 20 undiagnosed cases (Wu S L et al., Nat Comm 11, 4507, 2020). Clearly, the true scale of COVID-19 is bound by low molecular testing rates, which is primarily due to availability of key supplies and healthcare personnel, and low adoption rates of new viral gene targets.

At the time of infection and during its different stages, the first line of defense against SARS-CoV-2 is the immune response which includes the production of immunoglobulin M (IgM) and immunoglobulin G (IgG) antibodies in blood (Long, Q. X., et al., 2020, Nature Medicine, 26:845-848; Dispinseri, S., et al., 2021, Nature Communications, 12: Article 2670). In the process, IgM and IgG antibodies inhibit the viral load by binding to spike (S) and nucleocapsid (N) proteins of SARS-CoV-2 (Barnes, C. O., et al., 2020, Cell, 182(4):828-842; Ye, Q., et al., 2021, Frontiers in Immunology, 12: Article 719037). In symptomatic individuals, IgM antibodies appear in the early stages of viral infection and therefore are important indicator of the peak infection period. IgG antibodies, on the other hand, replace IgM antibodies after onset of symptoms and therefore are essential for long-term immunity and immunological memory (Li, P., et al., 2022, Journal of Clinical Laboratory Analysis, 36:e24080; Guo, L., et al., 2020, Clinical Infectious Diseases, 71(15):778-785). Remarkably, in asymptomatic individuals, the plasma/serum concentration of IgM antibodies is reported to be significantly higher than that in healthy people and not easily degraded within 7 weeks of infection. Whereas for IgG antibodies, the plasma/serum concentrations are reported to be above normal reference and increased with time in the 7 weeks of infection (Lei, Q., et al., 2021, Allergy, 76:551-561). Therefore, it becomes important to dynamically monitor plasma/serum IgM and IgG antibodies for efficient diagnosis and screening of SARS-CoV-2 infections [12], both in symptomatic and asymptomatic individuals (Long, Q. X., et al., Nature Medicine, 26:845-848; Lei, Q., et al., 2021, Allergy, 76:551-561; Jiang, C., et al., 2020, Clinical & Translational Immunology, 9:e1182). To establish this, antibody (serology) tests provide ideal route for early detection as well as identifying percentage of the population that is infected.

Due to the variability in the level of protective immunity among people, a large number of antibody-based detection and screening techniques are available in clinical laboratories, such as agglutination, enzyme immunoassay (EIA), and enzyme-linked immunosorbent assay (ELISA) (Chernesky, M. A., et al., 1984, Yale Journal of Biology & Medicine, 57(5):757-776). Agglutination is extremely fast (˜15-20 minutes) but requires large amounts of antigens for visible agglutination. EIA and ELISA, on the other hand, require the labeling of captured antibodies with fluorescent molecules or enzymes for the detection, identification, and quantification. All these techniques can be adapted for high-throughput and full-automation configurations with various degrees of success. However, the low number of antibody-antigen pairs that they can handle and inability to process samples at large scale possess a hurdle. Additionally, these techniques, though highly specific and sensitive, do not lend themselves to point-of-care applications due to necessity of equipment and skilled personnel.

Rapid point-of-care antibody tests are an alternative to laboratory antibody tests. Among these lateral flow assays (LFAs), with working mechanisms similar to standard pregnancy tests, are good examples that have been widely developed for colloid-based capture and detection of SARS-CoV-2 IgM and IgG antibodies from blood (Oh Y K et al., 2013, Lap Chip, 13:768-772). However, LFAs have their limitations, of which saturating the test (capture) lines with unbound (free) antibodies is particularly important since the phenomenon (known as “hook effect” in LFAs) leads to false negative results (Park J et al., 2017, Sensors & Actuators B: Chemical, 246:1049-1055). Colloid-based vertical flow assays (VFAs), on the contrary, are evolving alternatives that prevent the hook effect by sequentially delivering excess antibodies into the test zones, thus providing more reliable results (Park J et al., 2017, Sensors & Actuators B: Chemical, 246:1049-1055). VFAs are generally fabricated inside cassettes, which requires additional blood handling via capillary tubes. Here a novel hybrid (vertical-lateral-vertical) flow assay (HFA) is developed on an adhesive bandage-like device. The miniature device takes advantage of LFAs and VFAs working principles for friendly in-home. Here is described such a device for use with SARS-CoV-2 specific S and N proteins in a single test (FIG. 10). The device is amenable to other viral infections with simple adjustments.

### Design of a Hybrid Flow Assay (HFA)

The example HFA was designed in the form of an adhesive bandage for friendly in-home use (FIG. 10A), consisting of a front sample padding where blood is introduced and a transparent back readout where the detection zone is present. When a pinprick of blood (˜15 μL) is introduced into the pad (FIG. 10B), a red color appears at the control spot to confirm the validity of the results as negative (IgM−/IgG−) or positive (IgM+/IgG−, IgM+/IgG+, IgM−/IgG+) for SARS-CoV-2 infection (FIG. 10C).

The plasma processing area of the bandage (FIG. 10A) consists of a plasma separation membrane, conjugate pad, and nitrocellulose membrane vertically stacked onto one another, each with its own function (FIG. 10D). Following the introduction of blood, the plasma separation membrane, with ˜5 μm pore sizes, retains blood cells while allowing plasma to flow vertically into the conjugate pad (FIG. 10D, top panel). The SARS-CoV-2 IgM and/or IgG antibodies within the plasma then interact with dried AuNPs in the controllable spots of the conjugate pad (FIG. 10D, middle panel).

To prevent the competitive non-specific binding of other proteins in plasma, AuNPs are functionalized with SARS-CoV-2 S and N proteins specific to plasma IgM and IgG antibodies so that the targeted antibody-AuNP coupling is formed as a “lock and key”. For uniform horizontal flow and controlled delivery of plasma onto AuNPs, the conjugate pad is chemically modified to contain three hydrophilic spots (˜0.3 cm diameter) that are surrounded by hydrophobic barriers, one containing AuNP-IgE bioconjugates for validation of the assay and the other two containing AuNP—S and AuNP—N bioconjugates for capture of plasma IgM and IgG antibodies. Following interaction with AuNPs, antibody-AuNP complexes flow vertically to the detection zone by capillary forces.

The nitrocellulose membrane forms the detection zone of the assay (FIG. 10D, bottom panel), compartmentalized with three hydrophilic spots surrounded by hydrophobic barriers. To maximize analyte delivery and minimize cross-reactivity, the spot pattern (printed using a wax printer) was designed to match the spot pattern of the conjugate pad. The spots have biding surfaces coated with anti-IgE, anti-IgM, and anti-IgG secondary antibodies for specific capture and detection of IgE (control), IgM, and IgG antibodies, respectively (FIG. 10E). Validation of the results occurs when any of the antibody spots in the detection zone result in visible reaction (i.e., red color production) along with the control (FIG. 10F).

### Characterization of AuNP Bioconjugates

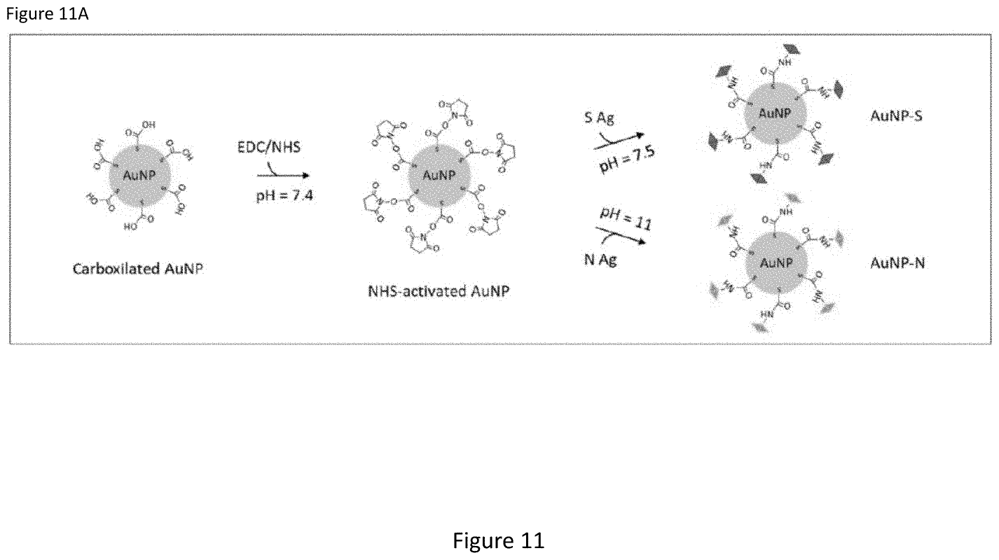

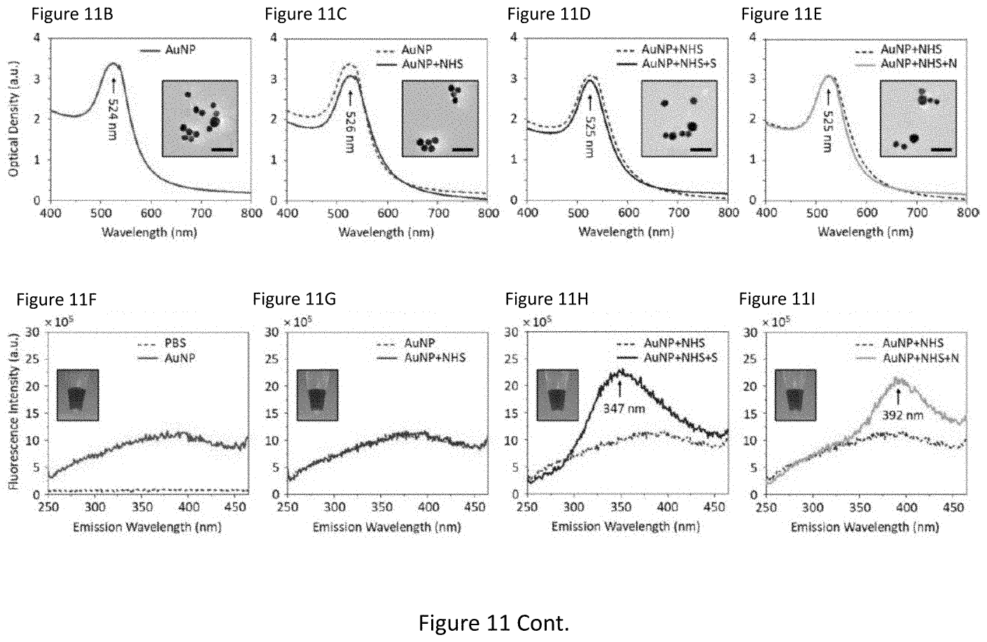

The first development step covered the optimization of bound SARS-CoV-2 spike (S) and nucleocapsid (N) antigens to AuNP surfaces in order to increase the specific capture of IgM and IgG antibodies. For this, carboxylated AuNPs, having high stability in liquid because of their double S—Au bonds, were modified with EDC/NHS chemistry to convert the surface carboxyl groups into more amino-reactive —NHS groups that would efficiently cross-link the S and N antigens (FIG. 11A).

Aggregation of the AuNPs was minimized by storage in potassium carbonate solution at a pH of 11. S and N proteins were introduced at a concentration of 1 mg/mL after readjusting the pH of the solutions to 7.5 pH and 11 pH for S and N proteins respectively. ˜1 nm shift in the final absorbance maxima (from ˜526 nm to 525 nm) verified successful AuNP—S and AuNP—N conjugation, supported by TEM images showing no signs of aggregation (FIGS. 11B-11E).

The chemisorption of S and N proteins to the NETS-activated AuNPs was validated by measuring their intrinsic fluorescence with fluorescence spectroscopy (FIGS. 11F-11I). Among the fluorophores in proteins, tryptophan is the dominant source of intrinsic protein fluorescence with its indole group responsible for the absorbance at ˜280 nm and emission at ˜350 nm (Ghisaidoobe, A. B., et al., 2014, International Journal of Molecular Sciences, 15(12):22518-22538). As such, during fluorescence measurements AuNP—S and AuNP—N bioconjugates were excited at 240 nm, and their emission was recorded between 250-450 nm. When excited, the emission of S and N proteins did not interfere with the plasmonic emission of AuNPs (˜423 nm at 308 nm excitation (Abdelhalim, M. A. K., et al., 2012, Journal of Nanomedicine & Nanotechnology, 3(3):1000133)). Thus, compared to carboxylated and NETS-activated AuNPs in FIGS. 11F and 11G, the emission peaks measured at ˜347 nm and ˜392 nm in FIGS. 11H and 11I were indicative of successful chemisorption of S and N proteins, respectively. It should also be noted that the emission of tryptophan is vastly affected by the polarity of its local environment, hydrogen bonding, and other non-covalent interactions, which could result in a 40 nm range for the peak wavelength differences (Ladokhin, A. S., et al., 2000, Analytical Biochemistry, 285(2):235-245). Therefore, the differences in the emission peak wavelengths of S and N proteins were likely the result of pH differences in AuNP—S and AuNP—N solutions (7.5 and 11 respectively).

Combined, the color of AuNP solutions remained unchanged as a function of surface activation (FIGS. 11F-11I), which further demonstrated their stability under optimized conditions. Moreover, in follow up experiments, AuNP—S and AuNP—N bioconjugates were additionally treated with 0.5% (v/v) protein-free blocking buffer to fill the unused binding sites on the surfaces, minimizing the non-specific binding of IgM and IgG antibodies to AuNP surfaces.

### Specificity in IgM and IgG Detection

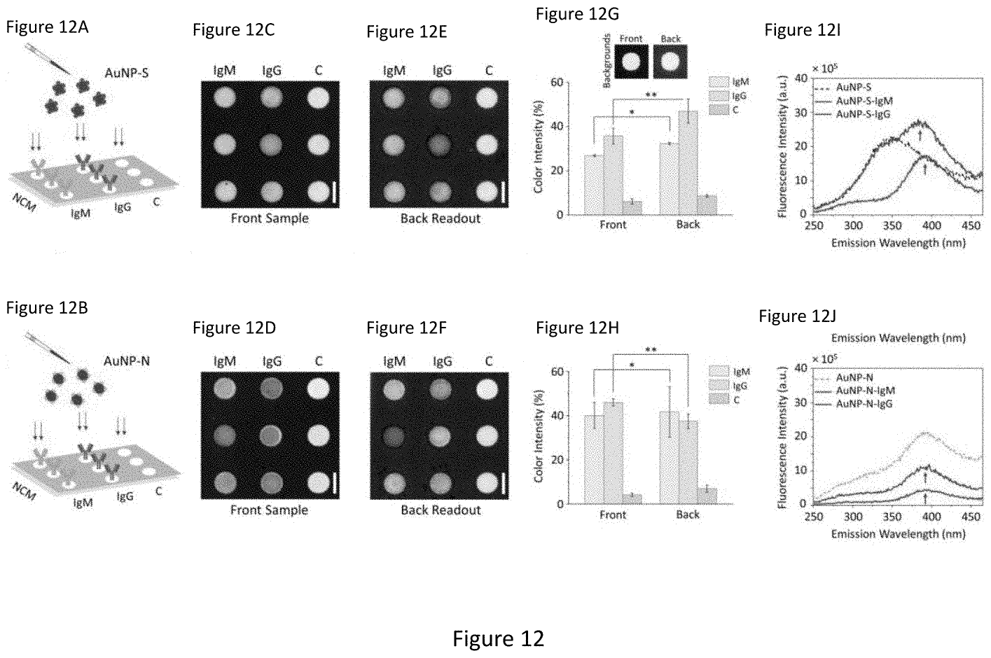

The third development step investigated the specificity of S and N proteins towards IgM and IgG antibodies (FIG. 12). Nitrocellulose membranes were first compartmentalized, using a wax printer, with 3×3 hydrophilic detection spots surrounded by hydrophobic barriers. IgM and IgG antibodies were then physically adsorbed within the 2×3 detection spots in pH 7 buffer and at 1 mg/mL concentrations for specificity measurements while the remaining spots were left untreated as controls to reveal the overall non-specific binding of S and N proteins to nitrocellulose fibers (FIGS. 12A and 12B. Following, uncoated surface fibers within the spots were further blocked with −0.5% (v/v) protein-free blocking buffer and 0.05% Tween-20 (v/v), after which 20 μL of AuNP—S and AuNP—N solutions were pipetted onto spots to allow coupling. Relative color intensities produced in the front (sample) and back (readout) of the nitrocellulose spots were analyzed and compared.

The color intensity difference visible to the eye revealed that, compared to controls, S and N proteins possessed high affinity for IgM and IgG antibodies (FIGS. 12C-12). Color profiles were uniform within front sample and back readout sides of the spots, further suggesting that IgM and IgG antibodies covered the surface of fibers in an oriented fashion (i.e., with two antigen-binding sites pointing towards the sample surface (Camarero, J. A., 2008, Biopolymers, 90(3):450-458). However, the degree of antibody coverage varied from side to side. For example, the coupling of S to IgM and IgG dominated the back readout by color intensity increases of ˜5% and 11%, respectively, while the coupling of N protein to IgM and IgG antibodies resulted in ˜2% increase and 8% decrease in the back readout color intensity, respectively (FIGS. 12G and 12H), attributed to heterogeneous pore size, distribution, and porosity of the nitrocellulose membrane. Together with the viscosity and surface tension characteristics of the fluid medium, this has been reported to affect the capillary flow of antibodies, influencing their non-homogeneous dispersion with the spots (Fridley, G. E., et al., 2013, MRS Bulletin, 38(4):326-330; Mujawar, L. H., et al, 2013, Analytical Chemistry, 85(7):3723-3729). It was also shown that when antibodies are allowed to bind to nitrocellulose fibers in acidic buffers (pH 2 and 3), they become 3 more resistant to removal upon treating the fibers with protein-containing blocking agents (e.g., dry milk, BSA) or non-ionic detergents (e.g., Triton X-100, Tween-20) compared to their binding in neutral buffers (Hoffman, W. L., et al., 1991, Analytical Biochemistry, 198(1):112-118). Therefore, during treatment of nitrocellulose fibers with Tween-20, already absorbed IgM and IgG antibodies may have become susceptible to removal, causing non-uniform distribution with the spots. Additional investigation is ongoing for the antibody removal dependency on both pH and buffer type used to bind the antibodies to nitrocellulose fiber.

The color intensity variations between the spots further revealed that S proteins are more reactive towards IgG antibodies than IgM antibodies with combined average color intensities of 41±4% and 30±1%, respectively, whereas for N proteins these values were 42±3% and 41±9% respectively, revealing similar reactivity towards both IgM and IgG antibodies. Small persistent sections of non-specific adsorption of S and N proteins to nitrocellulose fibers was also visually observed in all control spots (FIGS. 12C-12F). When characterized, the occurrences were evident by <10% color intensity increases relative to untreated front and back backgrounds (FIGS. 12G and 12H).

In general, the non-specific protein-to-fiber binding is either of hydrophobic nature (i.e., between hydrophobic portions of protein and carbon-containing nitrocellulose), or of electrostatic nature (i.e., between dipoles within proteins and dipoles of nitrate esters) (Fridley, G. E., et al., 2013, MRS Bulletin, 38(4):326-330; Van Oss, C. J., et al., 1987, Journal of Chromatography, 391(1):53-65). When combined with the large area of fibers and highly heterogeneous pore size, pore distribution, and porosity of nitrocellulose membranes, a great opportunity is provided for proteins to non-specifically adsorb to fiber surfaces and pore walls. Therefore, preventing these adsorptions is quite challenging when developing flow assays but also equally important for enhanced sensitivity, multiplexing, consistency, and reproducibility. To some extent, the non-specific protein-fiber binding is minimized by pre-treating the fiber surfaces with protein-containing blocking agents (e.g., BSA), and to some other extent, by chemically modifying them with protein repelling molecules (e.g., polyethylene glycol) (Shirshahi, V., et al., 2021, TrAC Trends in Analytical Chemistry, 136:116200; Zeng, C., et al., 1990, Analytical Biochemistry, 189(2):197-201). Nonionic detergents (e.g., Tween-20 in this study), on the other hand, are often used to efficiently saturate non-specific protein binding sites on nitrocellulose fiber surfaces (Batteiger, B., et al., 1982, Journal of Immunological Methods, 55(3):297-307). However, their blocking ability is partially removed during washing with water or buffer, immediately exposing the unblocked areas to protein adsorption (e.g., S and N proteins).

Moving forward, the binding of IgM and IgG antibodies to S and N proteins was additionally confirmed with fluorescence spectroscopy, where IgM and IgG antibodies (1 mg/mL) were allowed to interact with AuNP—S and AuNP—N bioconjugates and subsequently excited at 240 nm. Their emission was recorded between 250-450 nm. Compared to emission peaks of S (˜342 nm) and N (˜392 nm) proteins (FIGS. 11H and 11I), the emission peaks in FIGS. 121 and 12J represented successful binding of IgM and IgG antibodies. For S-IgM and S-IgG couplings, they were centered at ˜385 nm and ˜392 nm, respectively (FIG. 12I). Whereas for N-IgM and N-IgG couplings, they were centered at ˜390 nm (FIG. 12J).

### Conjugate Pad Design, Characterization, and Activation

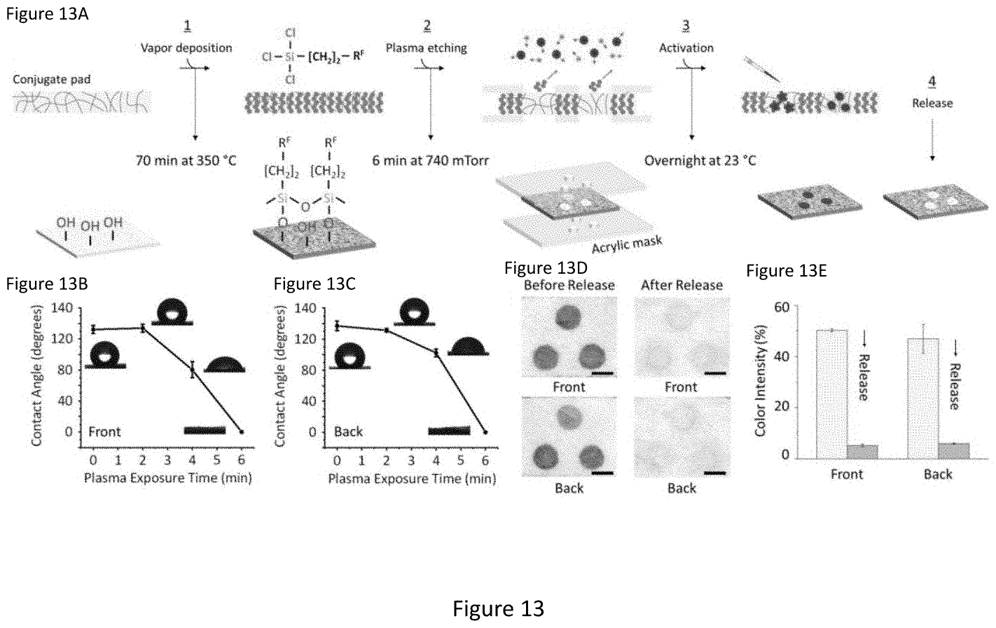

The conjugate pad is the upfront component in the HFA, where functionalized AuNPs are retained dry until the assay is performed for specific capture of target analytes. Upon conjugation, an enhanced spatial and temporal release of rehydrated AuNPs is essential for the analyte-AuNP complexes to flow towards the detection zone of nitrocellulose membrane with no additional control. Therefore, the conjugate pad should ideally preserve the functionality of dried AuNPs and exhibit low binding properties towards them. It is also equally essential that the release of analyte-AuNP complexes leads to less cross-reactivity in the detection zone for higher sensitivity. To address these needs, the third development step was first directed towards creating 3 hydrophilic spots (˜0.3 cm diameter each) within the conjugate pad so that AuNP-IgE (control), AuNP—S, and AuNP—N bioconjugates are compartmentalized. After which, the optimization of spots' pre-treatment conditions was followed for their efficient release upon rehydration (FIG. 13).

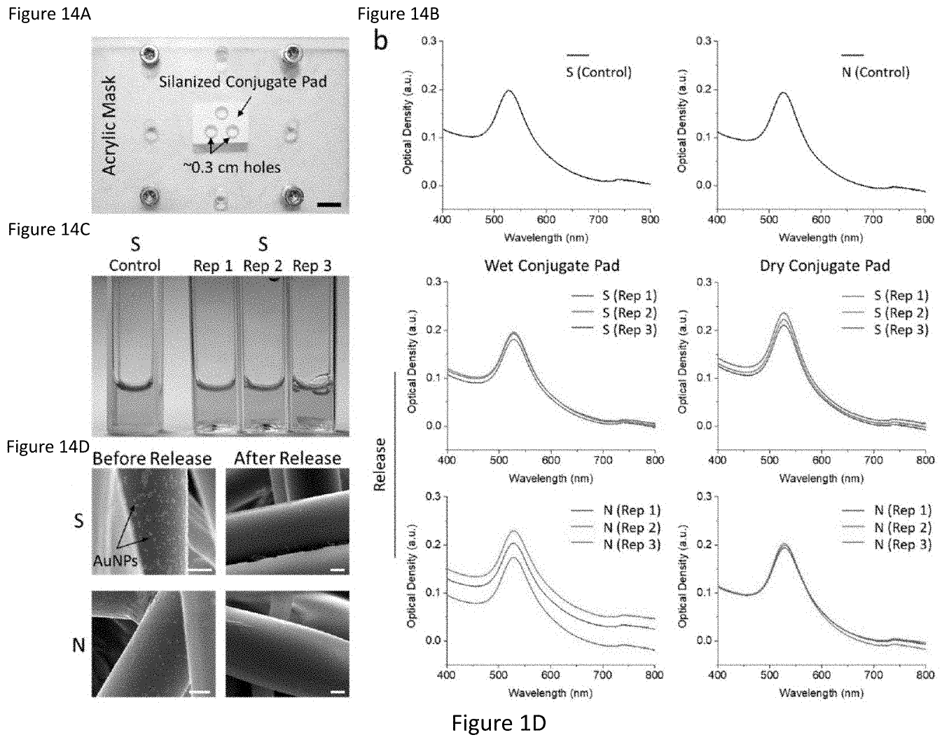

For successful completion of this step, the choice of conjugate pad was glass fiber due to advantages it brings, as it is inert, thus providing uniform and consistent flow characteristics, and can be chemically modified through silanization, thus leading to effective compartmentalization via etching. With this, the hydrophilic spots in the conjugate pads were created following the procedure described previously. As schematically summarized in FIGS. 13A and 13B, PFTS molecules were first vapor-deposited onto conjugate pads to generate a hydrophobic surface on glass fibers (step 1). Following, silanized pads were transferred in an acrylic mask with 3 distinct laser-cut holes (˜0.3 cm diameter each) on both sides (FIG. 14A). Oxygen plasma etching was then used to transfer the hole patterns onto conjugate pads by selectively removing the PFTS molecules from the fiber surfaces (step 2). As a result, the desired 3 hydrophilic spots were formed, where each spot was surrounded by hydrophobic barriers, and subsequently these spots were pre-treated for the activation with AuNPs (step 3) and their release upon rehydration (step 4).

During their vapor deposition, a vacuum is created on PFTS, which is allowed to evaporate and condense on the glass fiber surfaces. However, the quality (i.e., hydrophobicity and wettability) of the deposited PFTS layer strongly depends on the silanization time and temperature. A silanization time of 75 minutes at 350° C. temperature was found optimum for generating highly hydrophobic glass fibers with water contact angles of 132°±5° on the front and 137°±6° on the back sides of the conjugate pads (FIGS. 12C and 12D). Exposing the silanized fibers to oxygen plasma further revealed that the hydrophobic desorption efficiency was also good on the pads. As such, 6 min of plasma exposure turned, through oxidation of PFTS molecules to silanols (Si—O—H), the volume of conjugate pads into an entirely hydrophilic one, where the added water droplets immediately wetted both front and back sides of the pads (FIGS. 12C and 12D) (Liu, S. A., et al., 2017, Advanced Materials Interfaces, 4(11):1700027).

After forming the hydrophilic spots in the silanized conjugate pads, the next experiments covered pre-treatment for efficient release of rehydrated AuNPs. During optimization, absorbance measurements and SEM images taken before and after release were used as references. Together with visual inspection and quantitative color intensity analysis of spot images in the detection zones, results in FIGS. 13E and 13F and FIGS. 14B-14D revealed that sequentially pre-treating the hydrophilic glass fibers with 1% (v/v) BSA and 1% (v/v) Tween-20 facilitates over 95% release of dry AuNP-IgE, AuNP—S, and AuNP—N bioconjugates upon buffer exposure. This outcome was also comparable to their release from control undried (wet) conjugate pads (FIG. 14B). Here, BSA blocked the non-specific binding sites on glass fiber surfaces, whereas Tween-20 enhanced the release by “stripping” rehydrated AuNPs. Noteworthy, the hydrophobic barriers surrounding the hydrophilic conjugate spots were strong enough to prevent inter-zone mixing of AuNP-IgE, AuNP—S, and AuNP—N bioconjugates (FIG. 13E), thus allowing their complete vertical flow into the underlying detection spots (see FIG. 10D).

It has been reported that sequentially pre-treating fibers with 0.05% (v/v) Tween-20 and 20% (w/v) sucrose comparatively enhances the release of rehydrated antibodies from the polyester conjugate pads (Kaur, M., et al., 2022, Biosensors, 12(2):63). In the study, it was found that the approach generates highly viscose sample streams, which reduce the velocity of the vertical flow thereby increasing the interaction time of diffused antibodies with the membrane lying beneath conjugate pad (Kaur, M., et al., 2022, Biosensors, 12(2):63). Notably, the increased antibody interaction time could be beneficial in enhancing the overall sensitivity in the assay. Moreover, pre-treating the conjugate spots with sugars may additionally sustain the long-term stability and serological activity of dried S and N proteins (Tonnis, W. F., et al., 2015, Molecular Pharmaceutics, 12(3):684-694). Experiments utilizing this approach are ongoing.

### HFA Sensitivity Investigation

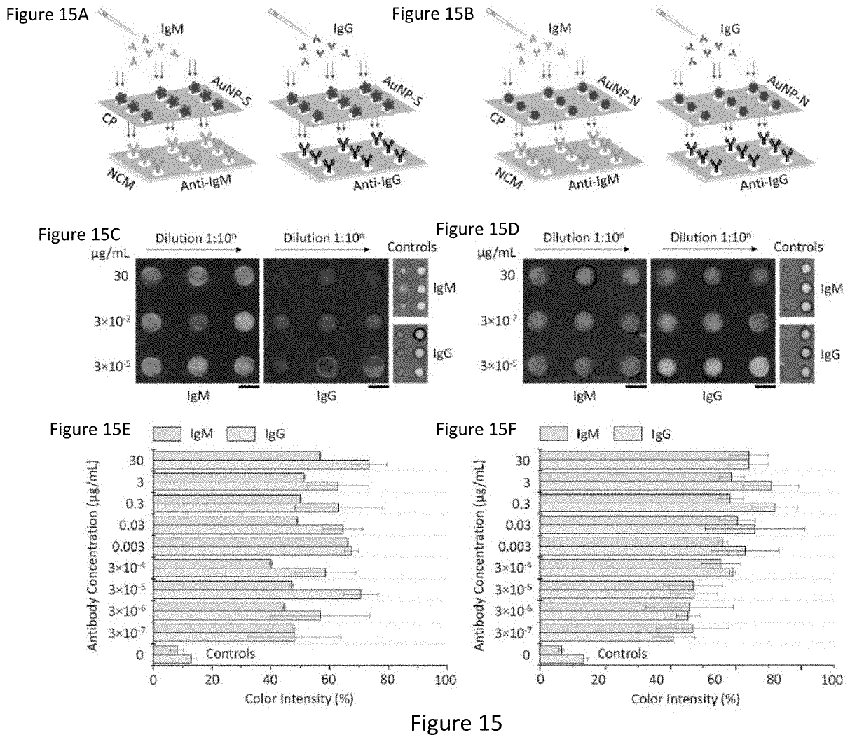

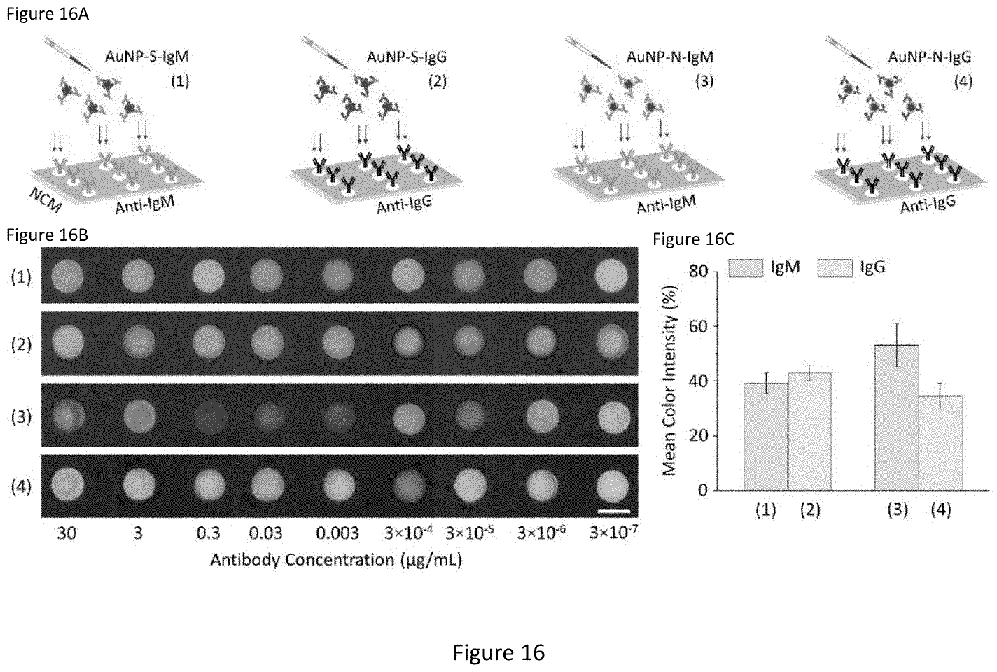

The reliability of the HFA directly depends on the specificity and sensitivity it offers. In the first two development steps, the specificity in HFA was promoted by functionalizing AuNPs with SARS-CoV-2 S and N proteins for the selective capture of plasma IgM and IgG antibodies. In the third development step the conjugate pad design and its pre-treating parameters were optimized for uniform and consistent release of dry AuNP—S and AuNP—N from upon rehydration. With these, the fourth development step focused on investigating the sensitivity of the assay (FIG. 15). In these experiments, conjugate pads and nitrocellulose membranes were first activated with 3×3 spots of dry AuNP—S/N bioconjugates and anti-IgM and anti-IgG capture antibodies, respectively, as described in previous sections. This was followed by pipetting concentrations of 30, 3, 0.3, 0.03, 0.003, 3×10-4, 3×10-5, 3×10-6 and 3×10-7 μg/mL IgM and IgG antibodies onto spots of conjugate pads, followed by gently pressing the conjugate spots against the underlying detection spots (FIGS. 15A and 15B). In another set of experiments, the same concentrations of IgM and IgG antibodies were initially allowed to couple with AuNP—S and AuNP—N bioconjugates in their respective buffers, after which resulting antibody-AuNP complexes were pipetted directly onto the nitrocellulose detection spots activated with anti-IgM and anti-IgG capture antibodies (FIG. 16). The purpose of the later experiments was to serve as control for the sensitivity of antibody-AuNP complexes released from the conjugate pad. In both sets, the color intensity produced in the back readout of nitrocellulose spots (detection zone) was taken into consideration for visual inspection and quantification.

Overall, the results in FIGS. 15C-15F revealed that the developed HFA is highly sensitive towards capture and detection of IgM and IgG antibodies. Excluding control color intensities in FIGS. 15E and 15F, which in average read <13%, for IgM and IgG detection through AuNP—S capture, the combined average color intensities read ˜50%±3% and 63%±10%, respectively, whereas their respective detection through AuNP—N capture resulted in average color intensities of ˜55%±7% and 59%±7%, respectively. These results were in agreement with the previous specificity results, confirming that S proteins are more reactive towards IgG antibodies than IgM antibodies, and N proteins are similarly reactive towards both. Visually, results further validated the uniform and consistent release of antibody-AuNPs complexes from the conjugate pads upon rehydration. Surprisingly, however, in the control experiments, the combined average color intensities produced by direct detection of AuNP—S-IgM/IgG read ˜39%±4% and 43%±3%, respectively, and those for AuNP—N-IgM/IgG read ˜53%±8% and 34%±5%, respectively. Here, the percent reduction in the color intensities was attributed to manually pipetting the solutions onto the spots which brought variations to the fluid contact angle, thus effecting the capillary flow rate (i.e., the passive wicking of fluids within the pores of the spots) and introducing shear stresses (Fridley, G. E., et al., 2013, MRS Bulletin, 38(4):326-330). As a result, the flow of the fluid is increased and interaction time of antibody-AuNP complexes with the capture antibodies in the spots is reduced, thus leading to significant reduction in their capture.

### HFA Functioning and Long-Term Storage Stability

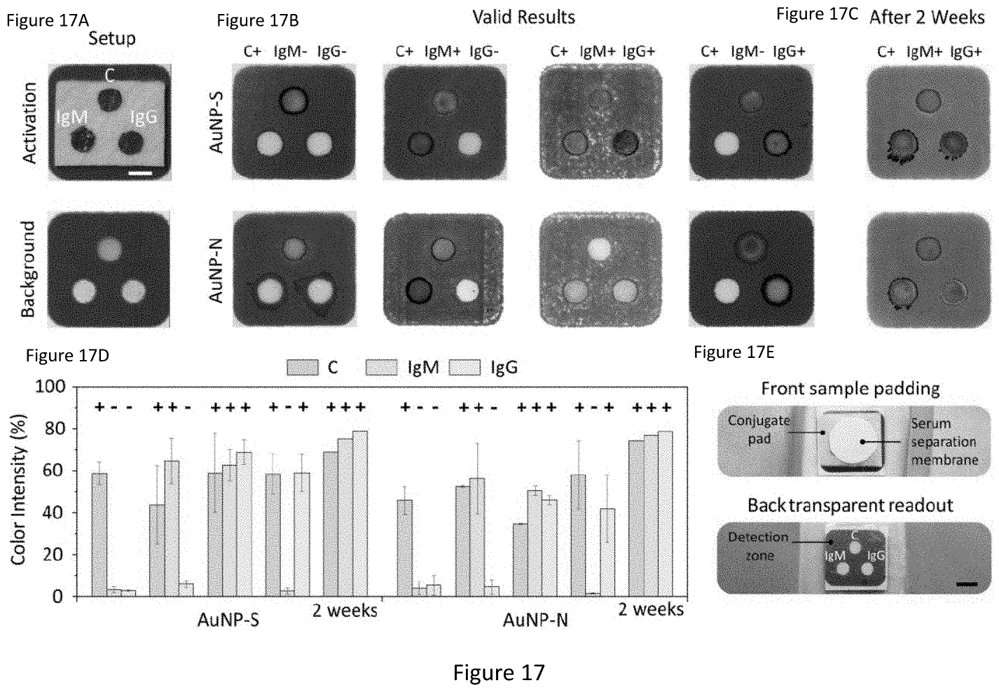

After preparing reliable and reproducible conjugate spots, which uniformly release antibody-AuNP complexes, and underlying detection spots, which capture antibodies efficiently and discriminately, the fifth and final development step included investigation of overall functioning and long-term storage stability of the HFA. Here, the conjugate pad, activated with dry AuNP-IgE (control), AuNP—S, and AuNP—N bioconjugates, was placed on top of a nitrocellulose membrane, activated with dry anti-IgE (control), anti-IgM, and anti-IgG capture antibodies. For controlled vertical fluid flow, the hydrophilic spots of the conjugate pad were aligned with the corresponding hydrophilic spots of nitrocellulose membrane (FIG. 17A). Four different antibody capture and detection conditions were then inspected, namely no spiked IgM and IgG (IgM−/IgG−), spiked IgM and no IgG (IgM+/IgG−), spiked IgM and IgG (IgM+/IgG+), and no IgM and spiked IgG (IgM−/IgG+), where spiked antibody concentrations remained the same for all cases (i.e., 3 μg/mL). To mimic their collective presence in plasma, IgM and IgG antibodies were further mixed at equal concentrations during IgM+/IgG+ investigation.

In all tested conditions, 15 μL of antibody solutions were directly pipetted onto plasma separation membrane, after which the membrane was gently pressed against the conjugate pad beneath it (FIG. 10D). The antibodies were then allowed to interact with rehydrated AuNP bioconjugates in the conjugate spots. Immediate color production took place within 3 min after the flow of antibody-AuNP complexes into the underlying detection spots, and reached its optimum by ˜15 min. Overall, results in FIGS. 17B and 17D revealed successful functioning of the HFA, both visually and quantitively. As such, in all valid results with positive outcomes (FIG. 10C), the presence of spiked IgM and/or IgG antibodies was evident with average color productions ranging from ˜35% to 60% in their respective detection spots. As desired, the overall cross-reactivity of spiked antibodies in non-designated detection spots was minimal, with <6% average color intensity production relative to background shown in FIG. 17A.

With these results, focus shifted to investigating the long-term storage stability of HFAs. For this, activated HFAs were first stored for 2 weeks in a desiccator. Following, IgM and IgG antibodies, spiked at equal concentrations (3 μg/mL) in buffer, were pipetted onto conjugate spots and allowed to produce color in the nitrocellulose detection spots. Results in FIGS. 17C and 17D revealed that the HFAs show excellent reproducibility for long-term storage stability of both AuNP—S and AuNP—N bioconjugates, as demonstrated by no loss of color in the detection spots (˜75-78% color intensities for IgM and IgG antibodies).

It is noteworthy to point here that, during the overall functioning of HFA, antibodies slip laterally in the conjugate pad due to highly hydrophobic glass fiber surfaces (Ho, T. A., et al., 2011, Proceedings of the National Academy of Sciences USA, 108(39):16170-16175). The pressure exerted by the finger, on the other hand, collapses the porous network of fibers, thus resulting in decreased porosity and permeability (Park, J., et al., 2016, Micromachines, 7(3):48). As a result, the lateral flow of antibodies naturally gets directed towards the three hydrophilic conjugate spots. More importantly, with increase in applied pressure, the capillary flow rate of antibodies also decreases within the conjugate/detection spots (Shin, J. H., et al., 2014, Biomicrofluidics, 8(5):054121). This, in turn, increases the time to passively wick the left dried volume within the spots, thus providing more time for antibodies to interact with rehydrated AuNPs and capture antibodies in the conjugate and detection spots, respectively. Accordingly, to minimize the ˜25% variation in the color intensity across the detection spots of the HFA, it is essential to quantitatively study the decreased permeability with respect to the decreasing porosity of the conjugate pad and nitrocellulose membrane upon applied pressure, and further provide additional means for the efficient delivery of antibodies into the conjugate spots, such as treating the surrounding hydrophobic glass fiber surfaces with various other surfactants. These investigations are ongoing.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A porous substrate-based diagnostic device, comprising:
a sheet of porous substrate having a thickness; and
a hydrophobic material patterned on the sheet of porous substrate;
wherein the hydrophobic material pattern extends through the thickness of the sheet of porous substrate such that the sheet of porous substrate comprises a plurality of hydrophilic regions divided by the hydrophobic material; and
wherein the hydrophobic material pattern defines a central hydrophilic sample region fluidly connected to at least one hydrophilic test region.

2. The device of claim 1, wherein the porous substrate is selected from the group consisting of paper, cloth, woven fabrics, non-woven fabrics, threads, yarns, perforated acrylamide polymer film, and perforated polyvinyl alcohol film.

3. The device of claim 1, wherein the hydrophobic material is selected from the group consisting of wax, silane, sioloxane, a fluororesin, and a silicone resin.

4. The device of claim 1, wherein the at least one hydrophilic test region comprises a capture molecule or probe selected from the group consisting of: antibodies, antibody fragments, antigens, aptamers, bacteriophages, proteins, nucleic acids, oligonucleotides, peptides, lipids, lectins, inhibitors, activators, ligands, hormones, cytokines, sugars, amino acids, fatty acids, phenols, and alkaloids.

5. The device of claim 1, wherein the at least one hydrophilic test region is arranged laterally, radially, or otherwise configured with the hydrophilic sample region.

6. The device of claim 1, wherein the at least one hydrophilic test region is arranged around the central hydrophilic sample region in a radial pattern for multiplex gene detection.

7. The device of claim 1, wherein the central hydrophilic sample region is configured to receive a liquid sample.

8. The device of claim 6, wherein the hydrophilic test region is loaded with at least one set of primers to detect the presence of one or more target nucleic acid molecules in the liquid sample.

9. The device of claim 8, wherein the hydrophilic test region is loaded with at least two sets of primers and pH-based loop mediated isothermal amplification (LAMP) reagents for use in a colorimetric LAMP assay.

10. The device of claim 1, wherein the hydrophilic test region is loaded with at least one functional particle to enhance the detection of one or more target molecules.

11. A vertical flow porous substrate-based diagnostic device, comprising:
a plurality of sheets of porous substrate stacked on top of each other, each sheet of porous substrate having a thickness and a hydrophobic material patterned on the sheet of porous substrate;
wherein the hydrophobic material pattern extends through the thickness of each sheet of porous substrate such that each sheet of porous substrate comprises a plurality of hydrophilic test regions divided by the hydrophobic material.

12. The device of claim 11, wherein the porous substrate is selected from the group consisting of paper, cloth, woven fabric, non-woven fabric, thread, yearn, perforated acrylamide polymer film, and perforated polyvinyl alcohol film.

13. The device of claim 11, wherein the hydrophobic material is selected from the group consisting of wax, silane, siloxane, fluororesin, and silicone resin.

14. The device of claim 11, wherein the at least one hydrophilic test region comprises a capture molecule or probe selected from the group consisting of: antibodies, antibody fragments, antigens, aptamers, bacteriophages, proteins, nucleic acids, oligonucleotides, peptides, lipids, lectins, inhibitors, activators, ligands, hormones, cytokines, sugars, amino acids, fatty acids, phenols, and alkaloids.

15. The device of claim 9, wherein a first sheet of porous substrate is configured to receive a liquid sample.

16. The device of claim 15, wherein at least one hydrophilic test region is loaded with at least one test antigen to allow detection of the presence of one or more antibody directed against the test antigen in the liquid sample.

17. The device of claim 16, wherein at least one hydrophilic test region further comprises one or more nanoparticles conjugated to the one or more test antigen.

18. The device of claim 16, wherein at least one hydrophilic test region further comprises one or more secondary capture antibodies.

19. The device of claim 15, wherein the first sheet of porous substrate comprises a filter membrane of thickness of 0.1-1 mm.

20. The device of claim 15, wherein the device is configured to allow the liquid sample to flow laterally between hydrophobic regions of adjacent sheets of porous substrates.

21. The device of claim 15, wherein the device is configured allow the liquid sample to flow vertically through hydrophilic regions across a sheet of porous substrate.

22. The device of claim 15, wherein the device is in the form of an adhesive bandage such that the first sheet is configured to contact a wound.

23. The device of claim 20, wherein the first sheet is configured with biodegradable porous microneedles.

24. A system comprising:
a) a first device comprising a porous substrate-based diagnostic device, comprising:
a sheet of porous substrate having a thickness; and
a hydrophobic material patterned on the sheet of porous substrate;
wherein the hydrophobic material pattern extends through the thickness of the sheet of porous substrate such that the sheet of porous substrate comprises a plurality of hydrophilic regions divided by the hydrophobic material; and
wherein the hydrophobic material pattern defines a central hydrophilic sample region fluidly connected to at least one hydrophilic test region; and
b) a second device comprising a vertical flow porous substrate-based diagnostic device, comprising:
a plurality of sheets of porous substrate stacked on top of each other, each sheet of porous substrate having a thickness and a hydrophobic material patterned on the sheet of porous substrate;
wherein the hydrophobic material pattern extends through the thickness of each sheet of porous substrate such that each sheet of porous substrate comprises a plurality of hydrophilic test regions divided by the hydrophobic material.
